(19) United States
(12) Patent Application Publication
(10) Pub. No.: US 2026/0128165 A1
(43) Pub. Date: May 7, 2026

(54) MACHINE VISION INSTRUMENT RECONCILIATION AND USAGE PREDICTION IN THE SURGICAL OPERATING ROOM

(71) Applicants: William E. BUTLER, Bristol, RI (US); Brian V. NAHED, Brookline, MA (US); Elizabeth LAMB, Bristol, RI (US); Hector ANDRADE-LOARCA, Munich (DE)

(72) Inventors: William E. BUTLER, Bristol, RI (US); Brian V. NAHED, Brookline, MA (US); Elizabeth LAMB, Bristol, RI (US); Hector ANDRADE-LOARCA, Munich (DE)

(21) Appl. No.: 19/381,238

(22) Filed: Nov. 6, 2025

Related U.S. Application Data
(60) Provisional application No. 63/716,886, filed on Nov. 6, 2024.

(51) Int. Cl. G16H 40/20; A61B 34/00; A61B 34/20; A61B 50/30; A61B 90/00; A61B 90/96; G06K 7/14; G06V 10/12; G06V 10/74; G06V 10/764; G06V 20/52

(52) U.S. Cl. CPC G16H 40/20; A61B 34/25; A61B 50/30; A61B 90/96; G06V 10/12; G06V 10/761; G06V 10/764; G06V 20/52; A61B 2034/2065; A61B 2090/0805; G06K 7/1413; G06V 2201/034

(57) ABSTRACT

A system for reconciling instruments in an operating room with sterile instrument ingress and egress staging zones, in which each zone is equipped with an imaging device for machine vision, bar code reading, or the like. The system utilizes a process for classifying and counting instruments based on imaging device data, and may include a graphical user interface with separate interface devices for sterile and non-sterile personnel. An active and real-time reconciliation log may be provided to track instruments and resources still in the sterile field, and hence at risk of remaining in a patient's wound. A subsequent egress sub-stage analysis for counting instruments used versus not used in the procedure may be performed and the results thereof aggregated to generate or update a predictive model to anticipate the likely instruments and other resources needed for future surgical procedures of the same type, facilitating efficient instrument selection and preparation.

### CROSS-REFERENCE TO RELATED APPLICATIONS

This application claims priority to U.S. Provisional Ser. No. 63/716,886, filed Nov. 6, 2024 and titled MACHINE VISION INSTRUMENT RECONCILIATION AND USAGE PREDICTION IN THE SURGICAL OPERATING ROOM, which is incorporated by reference herein in its entirety.

### FIELD OF THE INVENTION

The present invention relates generally to a system, method, and computer program product for reconciliation and/or usage prediction of instruments in a surgical operating room, and more particularly, using machine vision or other imaging techniques to identify, track, and predict usage of surgical instruments.

### BACKGROUND OF THE INVENTION

When a surgical procedure is scheduled, an instrument pick list is delivered to the surgery supply department. The pick list is a compilation of the number and types of sterile surgical instruments likely to be used in the procedure. A surgery supply department may aggregate the instruments in the pick list into a case cart to be sterilized and delivered to the scheduled operating room in advance of the surgical procedure. During the surgical procedure, various team members provide assistance to one or more operators who perform the procedure. While some team members may stay within sterilized environments, other team members may not. As an example, a surgical technician performs within the sterile environment while a circulating nurse and anesthesia team perform in the operating room, but outside of the actual sterile environment.

Prior to the surgical procedure, the operating room staff, primarily the surgical technician and the circulating nurse, open, inspect, and ensure that the correct type and number of surgical instruments are present. Counting surgical instruments and disposables such as sutures helps to reduce a number of risks to a patient, such as, for example, the risk of retaining an instrument in the patient's body (e.g., in the abdomen or the thorax). Retaining an instrument in the patient's body can be dangerous for any number of reasons, including a potentially imminent risk when an intraoperative MRI is used during the surgical procedure, where instruments must be removed from the magnetic field. If start and end counts of the surgical instruments and/or disposables match, then no instrument or disposable is assumed to have been retained in the body. If the counts do not match, then an instrument or disposable is considered to have been left in a body cavity of the patient. In such an event, the instruments or disposables are recounted, and if they still do not match, then the sterile field, including the body cavity of the patient, is searched for the retained instrument. Such searching may be assisted by an x-ray machine if the instrument is radio opaque (i.e., if it can be seen on X-rays).

In practice, many surgical instruments supplied in sterile form to an operating room are not actually used during the surgical procedure. Supplying surgical instruments that are not ultimately utilized can be costly and inefficient, as they require additional time to sterilize, understand, include, store, and account for them. Conversely, instruments that are required for the surgical procedure but not pre-supplied are often identified before or during the surgical procedure. In this scenario, operating room staff are required to retrieve the instrument(s) needed while the patient is under anesthesia, often with an open wound. Such delays can unnecessarily decrease the safety of the surgical procedure by lengthening the period of anesthesia needed and/or the length of time a wound or incision is left open and exposed. If surgical instrument(s) are not present yet urgently needed, then the delay can also adversely impact patient safety by increasing the risk of blood loss, infection, or other complication.

### BRIEF SUMMARY OF THE INVENTION

The present disclosure relates generally to a machine vision system and process for counting surgical instruments or disposables that ingress to a sterile or surgical field and that egress from the sterile or surgical field as a procedure nears completion, for maintaining detailed logs of the ingress and egress times of instruments, and for reconciling the ingress and egress machine vision logs. Additionally, a computerized predictive usage model may be generated based on the machine vision logs to increase the efficiency of future preparation and supply for sterile medical and surgical procedures.

Methods, systems, and computer program products are provided for managing a plurality of instruments in a sterile field. In accordance with one aspect of the invention, the system may comprise at least one imaging device; and a non-transitory computer readable storage medium coupled to the one or more processors and storing instructions thereon that, when executed by the one or more processors, cause the one or more processors to obtain one or more images of the plurality of instruments via the at least one imaging device upon ingress of the plurality of instruments into the sterile field; obtain one or more images of a set of the plurality of instruments via the at least one imaging device upon egress of the set of the plurality of instruments from the sterile field; generate an ingress inventory based on the one or more images obtained upon ingress of the plurality of instruments into the sterile field; generate an egress inventory based on the one or more images obtained upon egress of the set of the plurality of instruments from the sterile field; reconcile the ingress and egress inventories by comparing the ingress and egress inventories to determine whether or not one or more instruments were left in the sterile field; and output for display a result of the reconciliation to a user.

In certain embodiments, the at least one imaging device may include a first imaging device configured to obtain one or images of the plurality of instruments upon ingress of the plurality of instruments into the sterile field, and a second imaging device configured to obtain one or more images of the set of the plurality of instruments upon egress of the set of the plurality of instruments from the sterile field. Each instrument introduced into the sterile field may be identified based on the one or more images obtained by the first imaging device upon ingress of the instrument to the sterile field, and each instrument removed from the sterile field may be identified based on the one or more images obtained by the second imaging device upon egress of the instrument from the sterile field.

In certain embodiments, the one or more images obtained by the first imaging device upon ingress of a surgical instrument may be inputted to a first machine learning model trained to identify instruments from image data, and the one or more images obtained by the second imaging device may be inputted to a second machine learning model trained to identify instruments from image data. The first and/or second machine learning model may be trained to distinguish between visually similar instruments based on the presence or absence of a decorative discriminator applied to one or more of the instruments.

In certain embodiments, the at least one imaging device may further comprise a third imaging device configured to read scannable codes carried by the instruments, and each instrument introduced into the sterile field may be identified based on a scannable code read by the third imaging device upon ingress of the instrument, or based on one or more images obtained by the first imaging device upon ingress. Each instrument removed from the sterile field may be identified based on a scannable code read by the third imaging device upon egress of the instrument, or based on the one or more images obtained by the second imaging device upon egress.

In certain embodiments, a number of instruments in a package may be identified based on a scannable code on the package read by the third imaging device upon ingress into the sterile field, and/or based on one or more images obtained by the first imaging device upon ingress into the sterile field. An alert may be provided to a user when the number of instruments determined based on the scannable code differs from the number of instruments determined based on the one or more images.

In certain embodiments, the instructions, when executed by the one or more processors, may further cause the one or more processors to prompt a user for confirmation that the instruments identified by the system upon ingress or egress are counted only once. In certain embodiments, a graphical user interface may be displayed on a display screen with separate controls depending on the type of user, such as sterile users who remain in the sterile environment throughout the surgical procedure, and non-sterile users who need to step outside of the sterile environment. Separate input devices for sterile and non-sterile users may be provided to interact with the graphical user interface.

In certain embodiments, the second imaging device may be configured to obtain images of the instruments with and/or without an optical filter adapted to detect hemoglobin, other body fluid, or body tissue, and the set of instruments identified by the machine learning model during egress may be classified as used or not-used based on the presence or absence, respectively, of body fluid and tissue on the instruments.

In certain embodiments, a predictive model may be generated or updated to predict which instruments are likely to be used in a procedure based on a classification of instruments as used or not-used. The predictive model may comprise an ordinal logistic regression configured to provide a probability value corresponding to the number of instruments predicted to be used in a particular surgical procedure. The predictive model may additionally or alternatively comprise a large language model trained using a data set that includes usage data based on the classification of instruments used in a particular surgical procedure. In certain embodiments, the predictive model may comprise a multivariate logistic regression configured to predict the number of instruments used in a particular surgical procedure.

In accordance with another aspect of the invention, a method for managing a plurality of instruments in a sterile field comprises obtaining, using at least one imaging device, one or more images of the plurality of instruments upon ingress of the plurality of instruments into the sterile field; obtaining, using the at least one imaging device, one or more images of a set of the plurality of instruments upon egress of the set of the plurality of instruments from the sterile field; generating, using one or more processors, an ingress inventory based on the one or more images obtained upon ingress of the plurality of instruments into the sterile field; generating, using the one or more processors, an egress inventory based on the one or more images obtained upon egress of the set of the plurality of instruments from the sterile field; reconciling, using the one or more processors, the ingress and egress inventories by comparing the ingress and egress inventories to determine whether or not one or more instruments were left in the sterile field; and outputting, using a display device, a result of the reconciliation to a user.

In certain embodiments, the at least one imaging device used in the method may include a first imaging device configured to obtain one or more images of the plurality of instruments upon ingress of the plurality of instruments into the sterile field, and a second imaging device configured to obtain one or more images of the set of the plurality of instruments upon egress of the set of the plurality of instruments from the sterile field. The method may further comprise identifying, using the one or more processors, each instrument introduced into the sterile field based on the one or more images obtained by the first imaging device upon ingress of the plurality of instruments to the sterile field; and identifying, using the one or more processors, each instrument removed from the sterile field based on the one or more images obtained by the second imaging device upon egress of the set of the plurality of instruments from the sterile field. Identifying each instrument introduced and removed from the sterile field may including using the one or more processors to input the one or more images obtained by the first imaging device upon ingress to a first machine learning model trained to identify instruments from image data; and input the one or more images obtained by the second imaging device to a second machine learning model trained to identify instruments from image data.

At least one of the first and second machine learning models may be trained to distinguish between visually similar instruments based on the presence or absence of a decorative discriminator applied to one or more of the instruments. The least one imaging device may further comprise a third imaging device configured to read scannable codes carried by instruments. The one or more processors may be further used to identify each instrument introduced into the sterile field based on a scannable code read by the third imaging device upon ingress, or based on one or more images obtained by the first imaging device upon ingress, and to identify each instrument removed from the sterile field based on a scannable code read by the third imaging device upon egress or based on the one or more images obtained by the second imaging device upon egress.

In certain embodiments, the one or more processors may be further used to: determine a number of instruments in a package based on a scannable code on the package by the third imaging device upon ingress into the sterile field; determine a number of instruments in the package based on one or more images obtained by the first imaging device; and alert a user when the number of instruments determined based on the scannable code differs from the number of instruments determined based on the one or more images. The one or more processors may be further used to prompt a user for confirmation that the instruments identified upon ingress or egress are counted only once, and to display, on a display screen, a graphical user interface with separate controls for sterile and non-sterile users. The method may also comprise providing at least two separate input devices, including at least one input device for a sterile user and at least one input device for a non-sterile user.

In certain embodiments, the second imaging device used in the method may be configured to obtain images of the instruments with an optical filter adapted to detect hemoglobin, other body fluid, or body tissue. The method may further comprise classifying, using the one or more processors, the set of instruments identified by the machine learning model during egress as used or not-used based on the presence or absence, respectively, of body fluid and tissue on the instruments. The method may also further comprise generating or updating, using the one or more processors, a predictive model that predicts which instruments are likely to be used in a procedure based on a classification of instruments as used or not-used. The predictive model may comprise an ordinal logistic regression configured to provide a probability value corresponding to the number of instruments predicted to be used in a particular surgical procedure. The predictive model may comprise a large language model trained using a data set that includes usage data based on the classification of instruments used in a particular surgical procedure. The predictive model may additionally or alternatively comprise a multivariate logistic regression configured to predict the number of instruments used in a particular surgical procedure.

In accordance with yet another aspect of the invention, a computer program product comprises one or more non-transitory computer readable media having instructions stored thereon, wherein the instructions, when executed by one or more processors, cause the one or more processors to: obtain, using at least one imaging device, one or more images of a set of the plurality of instruments upon ingress of the plurality of instruments into the sterile field; obtain, using the at least one imaging device, one or more images of a set of the plurality of instruments upon egress of the set of the plurality of instruments from the sterile field; generate, using the one or more processors, an ingress inventory based on the one or more images obtained upon ingress of the plurality of instruments into the sterile field; generate, using the one or more processors, an egress inventory based on the one or more images obtained upon egress of the set of the plurality of instruments from the sterile field; reconcile, using the one or more processors, the ingress and egress inventories by comparing the ingress and egress inventories to determine whether or not one or more instruments were left in the sterile field; and output, using a display device, a result of the reconciliation to a user.

Various additional features of the systems and methods noted above may also be incorporated into computer program products, in accordance with the invention.

### BRIEF DESCRIPTION OF THE DRAWINGS

For a more complete understanding of the invention, reference is made to the following description and accompanying drawings.

FIG. 1 shows six images depicting different stages of a process for gathering and supplying sterile instruments for a scheduled surgical procedure, in accordance with an embodiment of the invention;

FIG. 2 is an image of an operating room table after a ventriculoperitoneal shunt revision operative procedure in which fourteen of one hundred and eighty six instruments were used and have blood on them, as indicated by arrows, in accordance with an embodiment of the invention;

FIG. 3 is a schematic diagram of an operating room table having a sterile field with sterile personnel and equipment, and machine vision ingress and egress staging areas for idempotent counting by human personnel and computerized actions, according to an embodiment of the invention;

FIG. 4 is a screenshot of a graphical user interface showing an ingress screen with dual users for capturing and counting sterile instruments delivered by non-sterile personnel to a sterile ingress staging area, according to an embodiment of the invention;

FIG. 5 is a classification tree diagram illustrating hierarchical relationships among different categories of surgical instruments, as used by a Machine Vision Ontology, according to an embodiment of the invention;

FIG. 6 is an image showing two example instruments (Adson and Adson-Barr forceps) whose machine vision discrimination is complemented by a decorative discriminator in the form of cyan tape, in accordance with an embodiment of the invention;

FIG. 7 shows three images depicting ingress staging of disposable suture packs containing sharp curved needles, in accordance with an embodiment of the invention;

FIG. 8 is a screenshot of an example graphical user interface screen for an example ingress stage, displaying an image with objects labeled and circumscribed by bounding boxes, in accordance with an embodiment of the invention;

FIG. 9 is a screenshot of an example graphical user interface screen for an example ingress stage, displaying a surgical instrument needing a retake due to overlapping instruments not recognized by the machine vision system, in accordance with an embodiment of the invention;

FIG. 10 is a screenshot of an example graphical user interface screen for an example ingress stage, displaying an example of instruments manually rearranged for proper recognition and counting by machine vision, in accordance with an embodiment of the invention;

FIG. 11 is a screenshot of an example graphical user interface screen for an example ingress stage, displaying an example of an ingress screen with an uncatalogued and therefore unrecognized instrument, in accordance with an embodiment of the invention;

FIG. 12 is a screenshot of an example graphical user interface screen showing an example reconciliation screen with a tabulation of running instrument counts passed into the sterile field via the ingress staging field, in accordance with an embodiment of the invention;

FIG. 13 is a flowchart of an example instrument ingress process in accordance with an embodiment of the invention;

FIG. 14 is a flowchart of an example instrument egress process in accordance with an embodiment of the invention;

FIG. 15 is a screenshot of an example graphical user interface screen showing an example egress stage, in accordance with an embodiment of the invention;

FIG. 16 is a screenshot of an example graphical user interface screen showing an example reconciliation stage two example screen with a tabulation of the ingressed surgical instruments from ingress stage one minus the egressed instruments from egress stage two, in accordance with an embodiment of the invention;

FIG. 17 is a screenshot of an example graphical user interface screen showing an example of egress counting of curved suture needles, in accordance with an embodiment of the invention;

FIG. 18 is a diagram containing an egress sub-stage configuration for usage data collection, showing an image of an egress sub-stage of instruments, a schematic diagram of an optical visible filter, an optical visible image, and a Near-Infrared (NIR) blood image, in accordance with an embodiment of the invention;

FIG. 19 is a flowchart of an example method in accordance with an embodiment of the invention;

FIG. 20 is a schematic diagram of a general computer system or information processing device that may be used with embodiments of the invention; and

FIG. 21 is a schematic diagram of a general computer environment that may be used with embodiments of the invention;

### DETAILED DESCRIPTION

To provide surgical instrument accountability, a system for idempotent classification and counting of sterile instruments at ingress to a sterile field and at egress from the sterile field is provided. Idempotency is the counting of each instrument once and only once at ingress and once and only once at egress. The invention may include a machine vision system for classifying and counting instruments that enter the sterile field and that exit the sterile field. It may also include a computer system with sterile and non-sterile interface tools for interacting with and supporting a process conducted by sterile and non-sterile personnel for the counting and reconciliation of instruments deployed and used. Additionally, the computer system may include a predictive computer model for anticipating and thereby helping prepare for instrument usage in surgical procedures.

As used herein, the terms “surgical procedure,” “surgeon,” “operating room,” “instrument,” “surgical technicians,” and “circulating nurses” have the following definitions. “Surgical procedure” refers to as any medical procedure performed in a sterile environment to reduce the risk of infection. “Surgeon” refers to the operator performing the surgical procedure. “Operating room” refers to a location that includes a sterile environment or field where the surgical procedure is performed. For example, some surgical procedures may be performed in a room of a hospital, while others may be performed in an outpatient setting in, for example, a specially equipped office of a health professional. “Procedure field” refers to an area where a procedure is performed, whether sterile or not.” “Instrument” refers to all potential tangible items to be identified, quantified, and supplied for a surgical procedure, whether or not the instrument is disposable. “Surgical technicians” refer to personnel who perform in the sterile environment. “Circulating nurses” and/or “anesthesia team members” refer to team members who perform outside of the sterile environment.

In certain embodiments, the invention may deploy separate machine vision systems for analyzing instruments that enter the sterile field and those that exit. The machine vision systems may be configured to monitor a sterile ingress zone with one or more cameras that capture images of instruments being transferred to the sterile field, and that monitor an egress zone to captures images of instruments being removed from the sterile field. An ingress barcode reader may be utilized for reading codes on various instrument packages.

In certain embodiments, one or more computer modules may be provided with software instructions executable by one or more computer processors for processing the images captured by the cameras at the ingress and egress zones, for machine classification and counting of instruments in the images, for maintaining logs of ingress and egress, and for reconciling the ingress and egress logs.

In certain embodiments, the machine vision classification may utilize an additional egress process for counting of the instruments used by the surgical team, as well as those which are not used. A statistical model of likely instrument usage in future similar surgical procedures may also be generated. This model may be used to generate the list of instruments to be supplied for sterile ingress in future, similarly scheduled surgical procedures.

In certain embodiments, a process may be followed that optimizes the safety and efficiency afforded by a machine vision instrument counting system coupled to a system for statistical prediction of instrument usage. One or more computer modules may be provided with software instructions that, when executed by a computer processor, provide distinct interaction tools for sterile personnel and non-sterile personnel. For example, in one embodiment, a computer mouse and keyboard may be provided for use by sterile personnel. In another embodiment, a sterile touch screen may be provided. These computer displays and input tools guide and support a process for maintaining surgical instrument accountability. Trained human clinical personnel may retain oversight and responsibility for surgical instrument accountability. However, the inventors realized that certain redundant and stereotypic activities that humans are less good at than machines may be performed by the systems and methods described herein. To promote safety and accuracy, the introduction and removal of surgical instruments may be performed in discrete stages to allow for strict overview, accountability, and sign-off by human personnel. In certain embodiments, the computer systems and methodologies described herein may allow for a reversionary mode, where the human personnel count the instruments by hand. In other embodiments, the process and method for maintaining surgical instrument accountability may be mostly or completely automated.

In an embodiment, the statistical model used by one or more computers may be a logistic regression that predicts the probability of usage of a given instrument as encoded by a stock-keeping unit (“SKU”) identifier based on a non-linear regression having a mixture of categorical and ordinal variables. It should be noted that the term SKU is used herein generically to refer to any type of unique identifier (e.g., a barcode number) associated with a specific instrument or other item in an inventory system. Examples of these input variables might include a diagnosis code, a planned procedure code, and one or more metrics of disease severity. For example, if the planned surgical procedure were for resection of a cancerous growth, then a metric of disease severity might include the cancer stage, such as the Tumor, Node, Metastasis system (TNM, https://www.cancer.gov/about-cancer/diagnosis-staging/staging). The logistic regression models may further be stratified by the identity of the surgeon, the identity of the hospital, and the identity of the patient, and the data may be divided into one or more groups before analysis.

In an embodiment, a Large Language Model (LLM) that is capable of dynamic vocabulary may be adapted and extended for instrument prediction. The LLM may be used to interpret and understand the different types of surgical instruments, surgical procedures, surgical notes and other information associated therewith, to create or update categorizations of the surgical instruments, and predictions as to which instruments will be used in particular surgical procedures. The LLM may be trained to analyze text, recognize semantic relationships, interpret content, and generate or update classifications, organizations, and summaries of instruments needed. The predictive model may comprise the large language model, and the large language model may be trained using a data set that includes usage data based on the classification of surgical instruments used in a particular surgical procedure.

While an LLM is given as an example, it will be appreciated that various components, steps, and functionalities of systems and methodologies of the invention described herein may be implemented using any suitable machine learning model or artificial intelligence technique capable of interpreting surgical instrument descriptions, surgical procedure descriptions, surgical notes, instruments counts from each surgical procedure, etc., and generating or updating categorizations of instruments and/or predictions of instruments to be used.

In this embodiment, the catalog of surgical instruments may be extended to include the SKU identifiers or other identifiers of the instruments. In the terminology of LLM customization, each instrument identifier may be incorporated into the LLM as a token. Each token may be associated with a feature vector. A large corpus of data may be gathered containing, for example, surgical instrument usage counts, surgical notes, and other medical records. The instrument embeddings may be learned during the LLM training, thereby enabling the LLM to understand the relationships between instruments and medical data, and thus, to predict the instruments used in a given surgical procedure, the timing for use of particular instruments in selected temporal phases of the procedure, and the spatial arrangement of instruments most consistent with their efficient handling by operating room personnel.

A number of LLM options may be utilized for these purposes. Some desirable LLM features include, for example, the allowance for a dynamic vocabulary and the smallness of LLM size. A compact LLM size enables two desirable features. First, it enables a single physical computer to host the LLM behind a hospital or operating room firewall, thereby protecting the privacy of patient information. Second, it enables a responsive graphical user interface arrangement for human personnel to oversee and manage the process of instrument count reconciliation over the duration of a surgical procedure. Examples of some open source LLMs that may be used are LLaMA (https://llama.meta.com/) and Mistral (https://docs.mistral.ai/). LLMs with specialized medical pretraining may also be utilized. The LLM training materials, as well as ongoing inputs and outputs therefrom, may be stored in database 218 of server 202 (further discussed below), external database 210, and/or on one or more client computer systems 206a, 206b.

In certain embodiments, the systems and methods described herein may be configured to address statistically expected errors by the machine vision system. By way of example, a comparatively serious error would be a total miscount error, e.g., where the number of instruments supplied or removed from the sterile field is not counted correctly. An important but less serious error is an instrument classification error where the machine vision system recognizes an object but misclassifies it. An instrument classification error would typically occur with two or more instruments with similar photographic characteristics.

In certain embodiments, the risk of an instrument total miscount error may be addressed by configuring the graphical user interface to present to a plurality of personnel (including sterile and non-sterile personnel) a computer screen with the identified objects annotated and labeled with bounding boxes. In an example embodiment, the plurality of human personnel may inspect the screens, and indicate whether or not they agree (e.g., via a separate “Accept” button). If all personnel click the “Accept” button, then an ingress (egress) stage is closed, and the classified and counted objects are added to a master reconciliation table. Instruments from an ingress stage may be added to the reconciliation table whereas those from an egress stage may be subtracted. At the conclusion of the procedure, the reconciliation table should display zero counts for all deployed instruments.

It will be appreciated that misclassification errors may arise from the machine vision analysis of instruments of different SKU identifiers that are similar if not nearly identical in size and shape when photographed. For example, two different sets of forceps may look similar, except for their respective inner grasping surfaces, which may have differences in texture. There is thus a need for a surgical instrument ontology (controlled vocabulary) that pivots off of the capabilities of a machine vision system. The present system and process may include a surgical instrument Machine Vision Ontology that hierarchically groups closely related instruments where the mutual misclassification by the machine vision system is comparatively more likely. In the system, instruments that are visually similar may have ontology terms that are closely grouped within the branches of the ontology, whereas instruments that are visually dissimilar will be remotely grouped along different branches of the ontology tree. In the example of a forceps, the higher term in the ontological hierarchy for the forceps may be “end-positioned lever fulcrum tool,” while the lower, more detailed terms in the ontological hierarchy might include a description of the grasping surface not easily discriminable or determinable by the machine vision system. With this Machine Vision Ontology, if at the conclusion of the surgical procedure the reconciliation counts for all subtypes of “end-positioned lever fulcrum tool” were to match and equal ‘0,’ then such a result would provide some assurance that the reconciliation was accurate within the limits of the machine vision system. From a patient safety standpoint, the important criterion would be satisfied, namely, that all forceps deployed were removed.

By way of example, surgical scissors and surgical snaps of similar sizes may appear similar to a computer vision system in that both have loops for a finger and move about a centrally placed fulcrum. An externally visible difference may be that surgical snaps have a locking mechanism to hold them shut when the levers are pushed together. In a visual instrument ontology, these both may be known as a “mid-positioned lever fulcrum tool.”

Another example of items that may have distinct catalog identifiers but similar features to machine vision are curved suture needles. Curved suture needles may come attached to different suture threads to be used on different tissues in different circumstances, but once detached, the metallic curved suture needles may have a similar appearance to machine vision such that they cannot be discriminated with satisfactory accuracy. However, from the perspective of safety of the patient, it is satisfactory if the total count of curved suture needles is accurate, not whether the different precise types of suture needles can be discriminated accurately and/or perfectly by machine vision.

In certain embodiments, a formal mapping may be carefully maintained between the visual instrument ontology and the catalog. At reconciliation, the final counts may be matched between the visual instrument ontology and the instrument catalog system. When the total number of ingress stages are aggregated, and the total number of egress stages are aggregated, the instrument counts of each item in both ontologies will be the same. The visual ontology system is thus designed to promote the speed and accuracy of visual instrument automated recognition across the ingress and egress stages.

In certain embodiments, the classification accuracy of similar appearing instruments may be enhanced by the application of decorative discriminators. An example of a decorative discriminator may be a piece of tape of specific size, shape, color, and/or position on a surgical instrument that distinguishes it in the machine vision system from an otherwise similar instrument. For example, continuing with the example of two pairs of forceps that may be of similar shape and size, one may have smooth tips and the other may have sharp tips, but this may be difficult to for a machine vision system to “see” or discriminate. To enhance classification accuracy, a piece of tape with a size, shape, color, and/or position known to the system may be applied to one of the forceps.

Surgical instrument packs are currently prepared, sterilized, and delivered to an operating room according to a system of fixed packing lists. The ability to predict instrument and resource utilization customized to a particular patient, procedure, and surgeon opens up other avenues for efficiency. As an example, the physical instrument packs may be made smaller as instrument usage is better predicted, potentially eventually enabling just-in-time instrument preparation where a small but complete instrument set is prepared and delivered given the particular knowledge of a particular planned procedure.

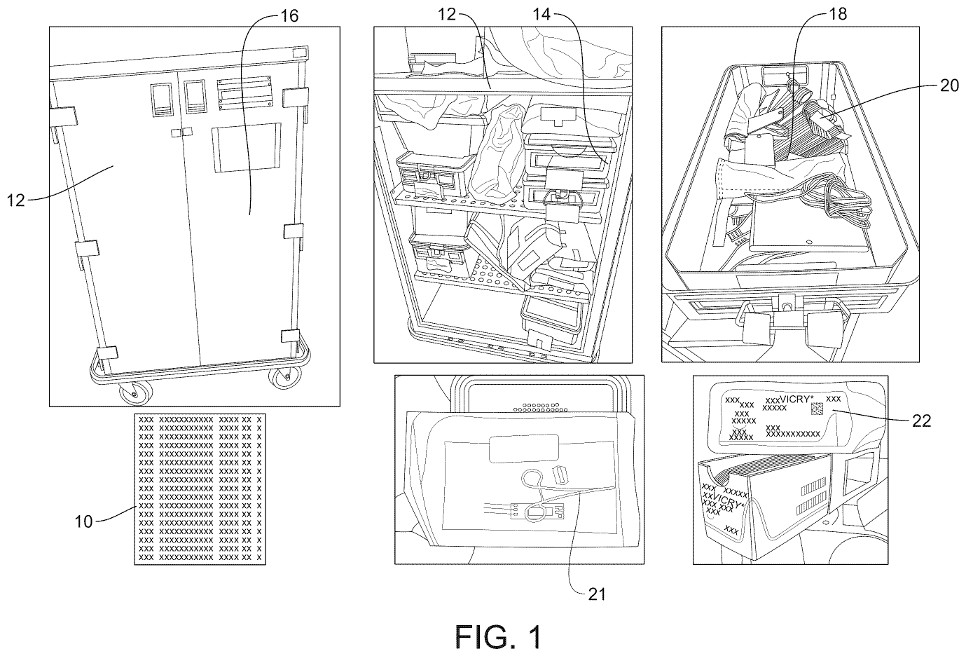

The six images of FIG. 1 depict different stages of a process for gathering and supplying sterile instruments for a scheduled surgical procedure. Initially, an instrument pick list 10 is generated and the collection of sterile supplies are loaded onto a case cart 12 to be transported to a scheduled operating room. Case cart 12 includes the predicted disposable and non-disposable surgical instruments typically used in the scheduled procedure. Prepackaged groups of instruments (instrument packs) commonly used together may be stocked, each in a sterile metal case. These instrument packs 14 are accessible when a front door 16 of case cart 12 is open. The outer surface of instrument packs 14 has a sterilization indicator, which changes color to indicate that instrument packs 14 and their contents have been sterilized. The open instrument pack 18 contains individual sterile instruments 20 along with a sterility color indicator.

Instrument packs 14 are placed into case cart 12 according to pick list 10. Additional instrument packs may be added, individually wrapped in sterile packaging, with a sterility indicator. The surgical team may request disposable sharp instruments (e.g., needles and/or scalpels) during the surgical procedure. For example, a sterile packing of a single instrument 21 may be requested if missing. Waiting for single instrument 21 to be found, sterilized, wrapped, and delivered can take fifteen to thirty minutes with a patient under anesthesia. As further described herein, the improved instrument tracking methodologies and predictive models of the present invention reduce the chance of missing such instrument(s) at the time of a surgical procedure. If, upon request, a suture pack 22 is supplied into the sterile field, the individual needles in pack 22 must be counted and logged upon opening of the sterilized pack before being placed into the sterile field. At the end of the procedure, all instruments are counted to ensure that every instrument is accounted for and not retained in the wound or body cavity.

Operations involving cavities (such as the thorax or abdomen) have the potential to retain an instrument. Therefore, all disposable and non-disposable instruments are counted prior to the conclusion of the procedure. Instruments added during the procedure are also counted and accounted for during and at the conclusion of the procedure. Similarly, procedures performed in the intraoperative MRI may also require strict accounting of instruments prior to starting and upon ending the procedure. Otherwise, the magnetic field from the MRI may move paramagnetic instruments hazardously toward the patient and personnel. If the operative site is not a body cavity, such as a procedure on the skin, then not all instruments need to be counted. However, sharp instruments such as scalpel blades and needles for sutures are counted to ensure that no instruments are retained.

In certain embodiments, unique identifiers may be placed on the package surface, whether case cart 12, instrument pack 14, or an individually wrapped instrument, as barcodes or other form of identifier or identifier code (also referred to herein as stock keeping units or SKUs). SKUs may come from different sources such as the Global Trade Item Number (GTIN, https://www.gs1.org/standards/id-keys/gtin) or the Universal Product Code (UPC, https://www.gs1.org/standards/barcodes/ean-upc). They may be read by machine and offer a source of instrument count reconciliation as discussed above. Different institutions (e.g., hospitals or facilities) may use different product identification systems, such as Global Trade Item Number (GTIN), Universal Product Code (UPC), or other schema for instrument naming and/or categorization.

In a machine-vision system for the recognition and counting of surgical instruments, a statistical error in object recognition and classification may be expected. Two categories of potential statistical errors are of distinct clinical significance. As noted above, one is an instrument classification error. The other is an instrument net count error. An instrument classification error occurs when the machine vision system recognizes an object but misclassifies it, which may occur for instruments having similar photographic characteristics. A net count error occurs when the machine vision system fails to recognize and count an instrument, regardless of how it classifies it. For example, if the machine vision system counts all instruments at ingress but fails to count one at egress, then the surgical team may not be alerted by the system to the possibility of a retained instrument in the patient's body. Example embodiments of the invention provide a system for handling these errors and reducing their impact on the safety and efficiency of surgical procedures.

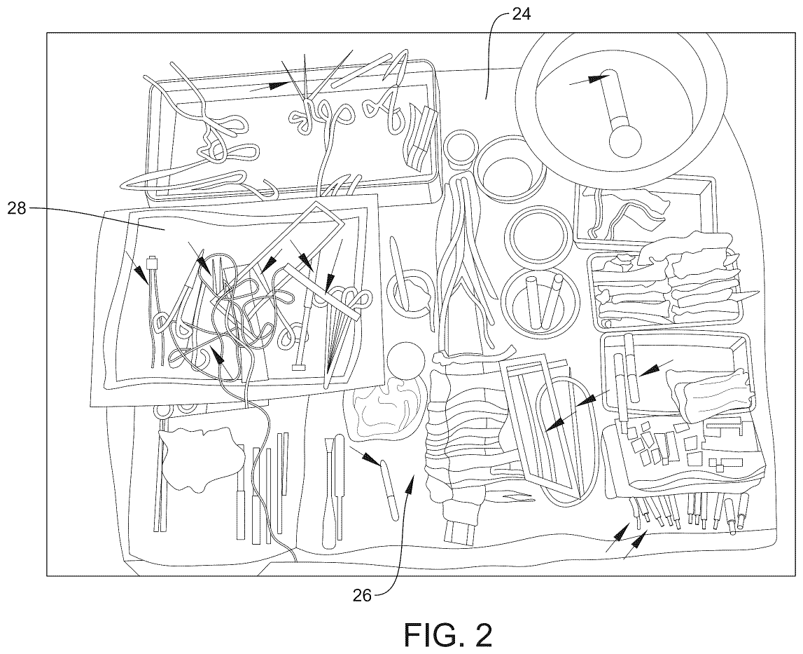

FIG. 2 is an image of an operating room table 24 taken upon completion of a surgical procedure (e.g., a ventriculoperitoneal shunt revision operative procedure). Table 24 has about one hundred eight-six (186) instruments 26. Of those, about fourteen were used, as indicated by one criterion of having blood on them, shown by arrows 28. It is wasteful that 92% (186−14)/186 of supplied instruments were not used.

If a surgical instrument is required that is not available in the pack, it may cause an unnecessary delay while the patient is under general anesthesia, prolonging the procedure, as the needed instrument is first identified as missing, then requested, and ultimately obtained and delivered from the surgical instrument supply room. Surgical instruments that are included but not needed or used add to the inefficiency and cost of the procedure, because those instruments need to be counted before and after the procedure, and must be cleaned, stored, sterilized, and repackaged for the next surgical procedure despite never being used. Therefore, in certain embodiments, the machine vision instrument classification and counting system may be configured to classify and count those instruments used and not used by the surgical team. In certain embodiments, instrument usage data may be analyzed to create a statistical predictive model, aiming to accurately predict the specific instruments needed for a given patient, diagnosis, surgeon, specialty, and planned surgical procedure. In certain embodiments, a system may be implemented that includes methods driven by a predictive statistical model for efficient and accurate surgical instrument selection, packaging, and supply for a scheduled surgical procedure.

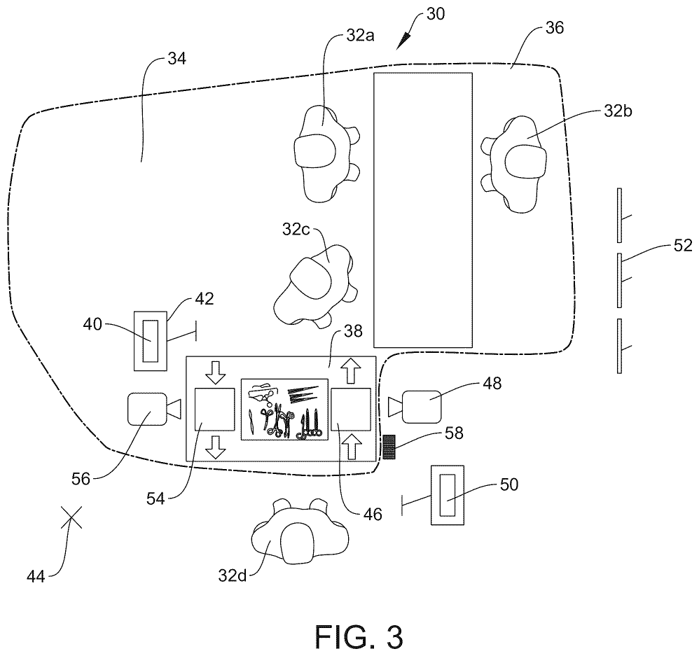

FIG. 3 is a schematic diagram of a physical arrangement 30 of personnel (32a, 32b, 32c, 32d) and various objects in an operating room during a surgical or other sterile procedure. In the operating room, a sterile vicinity or sterile field 34 is shown bounded by a dashed curve 36. Sterile field 34 within dashed curve 36 includes torsos and arms, wrists and hands of sterile personnel (32a-32c), and everything they might touch, including the exposed part of a patient, the tabletops (e.g., instrument table) 38, and computer equipment 40 with sterile covers 42.

Using sterile covers 42 maintains a sterile environment and prevents infection. For example, when an x-ray machine is briefly brought into the operating room it is placed in a sterile cover. These covers act as a barrier, ensuring that the equipment does not contaminate sterile field 34 or the surgical area, thus protecting the patient from potential infections. Instrument table 38 is attended by a surgical scrub technician 32c. Sterile operating personnel 32a, 32b carry out the procedure. A circulator nurse 32d bridges the sterile 34 and non-sterile 44 zones. Circulator nurse 32d removes the non-sterile outer packaging of sterile instruments and other sterile supplies (disposable and/or non-disposable) to be placed onto instrument table 38.

As further shown in FIG. 3, arrangement 30 also includes a sterile ingress staging zone 46 where countable sterile supplies and instruments entering sterile zone 46 are placed, identified, and accounted for. Images of items placed in ingress staging zone 46 may be captured by an ingress camera 48 for recognition and counting by machine vision. In certain embodiments, an idempotency system may be utilized to assure that sterile instruments transferred across ingress staging zone 46 are counted only once. In an example embodiment, idempotency is achieved and verified by a process that is supported by a network of computerized graphical user interfaces. For example, a given snapshot with objects recognized and counted may be jointly inspected and approved by more than one person, e.g., by surgical scrub technician 32c and the circulator nurse 32d, and internal quality metrics may be applied in an automated fashion by the computerized machine vision system. The sterile personnel interact with a sterile ingress computer display and interface system 40 within the sterile vicinity 34 that is preferably covered with a clear plastic drape (i.e., a sterile cover) 42 so that the keyboard and mouse may be used while maintaining sterile technique. After objects are counted in ingress zone 46, they may be deployed in the sterile procedure. The non-sterile personnel interact with an egress computer display and interface system 50. For a given point in time in the procedure, the net reconciliation status (e.g., the aggregate instruments ingressed minus egressed) may be displayed on a reconciliation screen 52, which may be positioned in an elevated position for all personnel to monitor. For the egress process, an egress staging zone 54 is monitored by an egress camera 56.

In certain embodiments, further automation may be utilized. Rather than still images based on human prompting of the ingress and egress staging zones, continuous video of sterile field 34 may be provided. The video footage may be fed continuously to a machine vision software process in a computer that detects in real time the identities of objects and their ingress to and egress from sterile field 34 (e.g., sterile ingress zone 46). The results may be used to update a reconciliation table for display in the various graphical user interfaces presented to users on the surgical team.

In certain embodiments, the ingress staging zone 46 may have an ingress bar code reader 58. This allows the logging of barcoded objects across ingress staging zone 46. In one example suture needle packages, which contain a declared and expected number of needles, are used. The needle package may have a barcode that connects to a catalog number that specifies the number of needles the manufacturer declares to have placed in the needle package. The needles may be counted in the staging areas as they enter and exit sterile field 34. The needle ingress counts may be matched to the expected counts as per their respective catalog information. If there a discrepancy is determined, then the manufacturer may be notified subsequently to assist its internal quality control system. At the end of the surgical procedure, if the ingress and egress counts match, then it may be inferred that none of the needles are retained in a surgical field at the end of the surgical procedure. If the ingress and egress needle counts do not match, then an exception may be noted by system software to alarm and request human attention, via a graphical user interface across both egress computer 50 and sterile ingress computer 40.

The accountability of instrument usage in an operating room spans across two work contexts, the sterile and the non-sterile contexts, and three tasks, ingress accountability, egress accountability, and ingress vs. egress reconciliation. While the process may be unified, each of these contexts and tasks has specific criteria that may be supported by a particular graphical user interface. In certain embodiments, each task may have a dedicated computer display screen, and each screen may have graphical user interface widgets dedicated for use by sterile and non-sterile personnel. Sterile personnel, for example, may have a computer system with a screen, keyboard, and mouse with a sterile covering so that sterile personnel may use it freely during the procedure. An example of this is shown in FIG. 3 where a sterile sterile-ingress computer 40 is provided for use by personnel, and a non-sterile egress computer 50 is provided for use by other personnel.

In addition to separate computer systems, one sterile and one non-sterile, in certain embodiments, each computer system may have hardware configured to display a graphical user interface for personnel to act and view the actions of other personnel. Such graphical user interfaces may support a process that includes joint oversight and agreement of instrument recognitions and counts by the machine vision system. For example, in a single screen as rendered by the graphical user interface, the color and/or shading of the arrow mouse pointer on the screen may be dependent on whether it appears in the sterile or non-sterile version of the graphical user interface so that a third person observer upon inspecting the screen may know whether a given mouse pointer is being manipulated by the sterile or the non-sterile personnel.

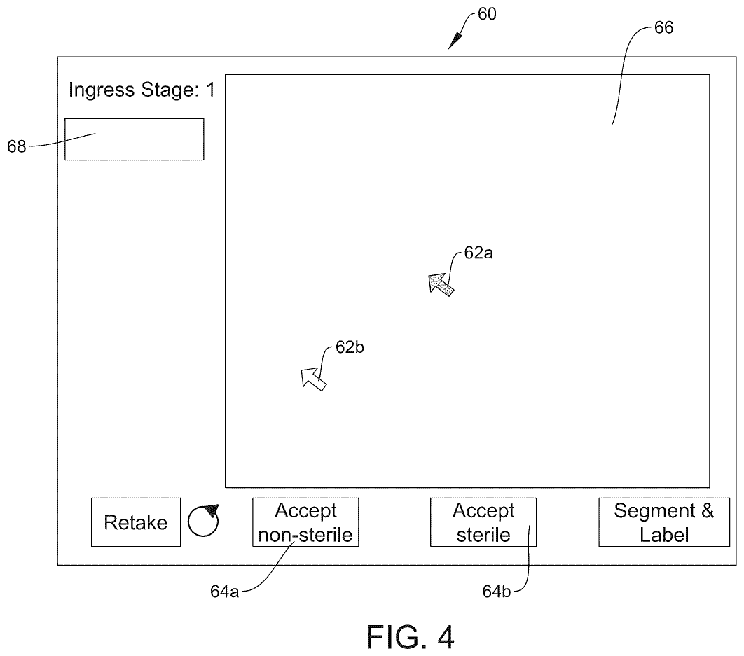

An example of such a joint screen and graphical user interface arrangement is illustrated in FIG. 4, which is a screenshot of a first graphical user interface 60 with two different mouse arrows 62a, 62b and two different user interface buttons 64a, 64b. An ingress image screen 66 is configured to display video of sterile ingress staging zone 46 may be shown. Mouse icons 62a, 62b controlled by each hardware user interface mouse may be correspondingly shaded and/or colored differently to clarify which personnel are taking which action(s). For example, a mouse covered with a sterile wrap may control a specific sterile mouse icon 62a that is shaded on screen 66 to indicate the particular user who is controlling it. Likewise, the mouse controlled by a non-sterile person/user may correspond on screen 66 to non-sterile mouse icon 62b, which may be shaded and/or colored differently than mouse icon 62a.

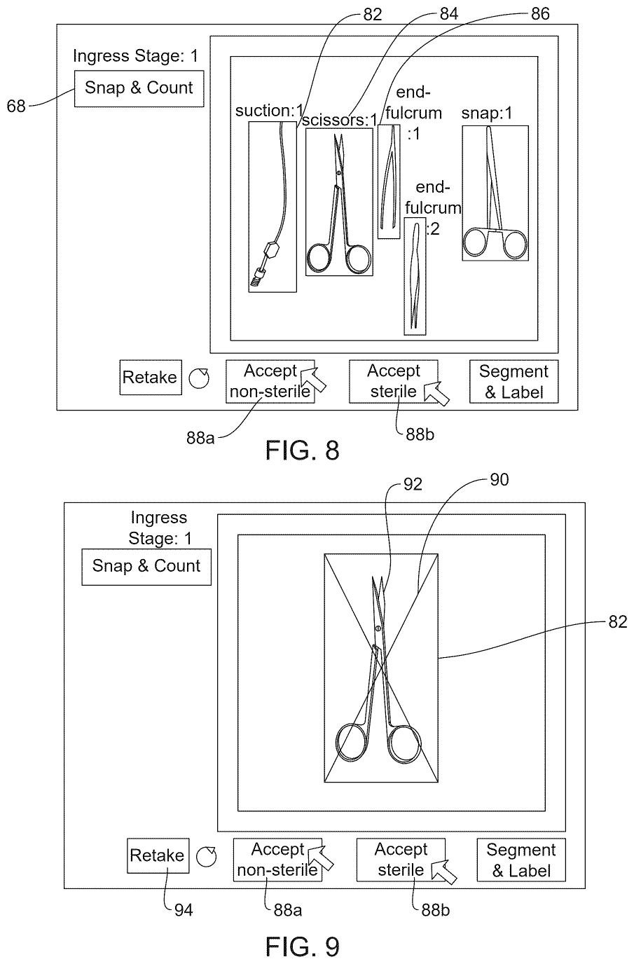

In certain embodiments, when a snap and count button 68 is clicked by any personnel, a still shot image (e.g., a photograph) may be taken of sterile ingress zone 46 and sent to the machine vision system for recognition and counting. In certain embodiments, separate user interface buttons may be provided for different personnel, which when clicked, indicate that respective personnel have inspected and approved the machine vision recognition and count. As with mouse icons 62a, 62b, user interface buttons 64a, 64b to be clicked may be correspondingly shaded differently. For example, sterile accept button 64b may be shaded to match the shading of the sterile mouse icon 62a. Similarly, non-sterile accept button 64a may be shaded to match the shading of the non-sterile mouse icon 62b. The segregation of sterile and non-sterile user interface widgets supports the discipline of unanimity in accepting and inspecting usage, instrument identification, and counts by the computerized machine vision system. In certain embodiments, an ingress stage may be treated as closed only when both agree and signal this by clicking their respective “Accept” graphical user interface buttons. Examples of these screens in particular circumstances are shown in FIGS. 8, 15, and 16. The instrument labels in these graphical user interface screens may draw vocabulary from the Machine Vision Ontology, further described below.

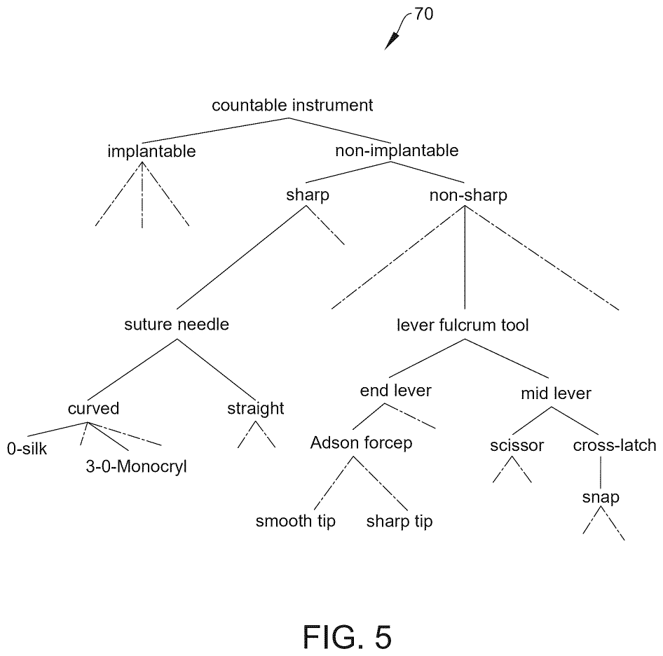

Referring to FIG. 5, a classification tree diagram 70 illustrates hierarchical relationships among different categories of surgical instruments, as used by an example Machine Vision Ontology. Classification tree diagram 70 shows example branches of the Machine Vision Ontology. This controlled vocabulary is tuned for the needs of accurate surgical instrument machine vision. In certain embodiments, the machine vision system may undergo training in which it is given images (e.g., photographs) having objects of known type and location. The type is specified as a string drawn from the Machine Vision Ontology. After a sufficient number of examples of an object, the machine vision system learns to recognize it and applies a bounding box about it, in a processed version of a given test photograph. Items in the Machine Vision Ontology are then mapped by a table to items in the SKU catalog. The visible characteristics of an instrument evident in a photograph distinguish instruments from each other in machine vision. Thus, a high degree of statistical accuracy in the machine vision system is preferable. As discussed above, certain instruments may be provided that are similar in appearance in a photograph to other instruments, but have different functions and features not captured by the machine vision system. For example, a mid-positioned lever fulcrum tool may have a locking mechanism to hold it shut when the levers are pushed together.

The ontology is designed to cluster in a hierarchy instruments with similar visual features. Statistical recognition error may be greater down toward the leaves of the hierarchical ontology, but for clinical safety purposes, statistically accurate counting at higher levels of the hierarchy may meet needs. At the highest level of detail are the leaves of the Vision Ontology. A particular item may be specified with sufficient detail that it is attached to an SKU catalog identifier. As shown, at the top of example classification tree diagram 70, the label ‘countable instruments’ is used. The first categorization is ‘implantable’ versus ‘non-implantable.’ Under the ‘non-implantable’ category, the instruments are categorized as ‘sharp,’ such as a ‘suture needle’ versus ‘non-sharp,’ such as a ‘lever fulcrum tool.’ Under ‘suture needle,’ the instruments are categorized as ‘curved’ versus ‘straight,’ and under ‘lever fulcrum tool,’ as an ‘end lever’ or a ‘mid lever.’ Under ‘curved,’ the instruments are categorized as ‘0-silk’ versus ‘3-0-Monocryl.’ Under ‘end lever,’ an ‘Adson forcep’ is categorized, which can have a ‘smooth tip’ or a ‘sharp tip.’ Under ‘mid lever,’ the instruments are categorized as a ‘scissor’ and a ‘cross-latch,’ which can include a ‘snap.’

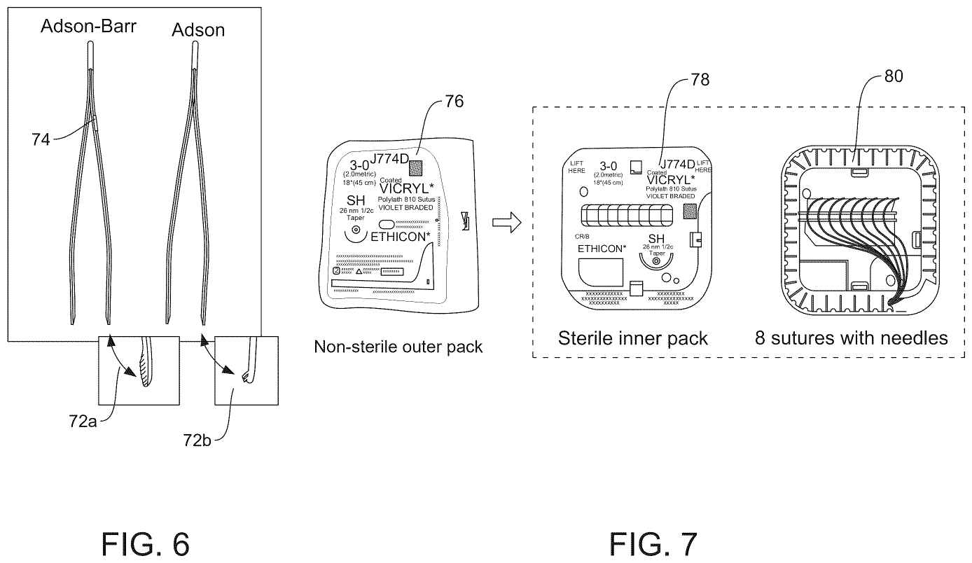

FIG. 6 shows an example use of the hierarchical Machine Vision Ontology and of a decorative discriminator in the example of two forceps with a nearly identical visual appearance. In the Machine Vision Ontology, the forceps fall under the hierarchical category termed “end-fulcrum lever tools.” This name is chosen for the Machine Vision Ontology because it describes the basic structure to be perceived by a machine vision system. The two forceps are similar to each other except for a tip difference 72a, 72b. Tip 72b is of an Adson forceps, and has a toothed shape for pinch-grasping, whereas tip 72a is of an Adson-Bar forceps, and has a rough but non-sharp surface for grasping softer tissues. These differences may not be perceived by a machine vision system, but for clinical safety purposes, they do not necessarily need to be distinguished so long as the total count of end-fulcrum lever tools is the same at final reconciliation. For those particular instances where the machine vision distinction of similar-appearing instruments is desired, for both safety and efficiency purposes, a decorative discriminator may be used to distinguish the instruments. In this scenario, decorative discriminator is a piece of cyan tape 74. A decorative discriminator may be selected to satisfy a threshold of machine vision information augmented to the basic shape of the instrument to enable the distinction by machine vision among otherwise similar-appearing instruments. In certain embodiments, each instrument may be configured with a scannable code scannable by ingress camera 48, egress camera 56, or one or more additional cameras.

Special attention may also be applied to the accountability of sutures because they come supplied with sharp needles, which may be particularly injurious if left in a body cavity and to personnel if misplaced. FIG. 7 shows combined use of the barcode reader and machine vision in a specialized ingress procedure for suture packs having needles, an embodiment of the needle pack example discussed above with respect to FIG. 3. An example sealed suture package 76 contains sutures with needles to be passed idempotently across ingress staging area 46. This outer package is not sterile. It is a peel pack that is opened carefully by circulator nurse 32d so that sterile inner suture package 78 may be passed cleanly onto ingress sterile field 46. This holds sterile suture dispensing package 80 where curved needles are positioned for grasping by the metallic needle holders.

Sealed suture package 76 and sterile inner suture package 78 both have a dot matrix barcode that encodes the Global Trade Item Number (GTIN). The system may read the barcode (e.g., with a barcode reader) and automatically look up the contents of the package. For instance, the GTIN in this example identifies the package as containing eight sutures with needles. When the contents are deployed across the staging zone to the sterile field, the contents may be added in the software process to the running reconciliation table.

Circulator nurse 32d opens sealed suture package 76 and scrub tech 32c takes possession of sterile inner suture package 78. It is opened to reveal the eight sutures with needles in sterile suture dispensing package 80. This may be inspected by machine vision in the ingress staging zone and the number of needles may be counted by machine vision. If the number of needles does not match the corresponding number by lookup of the GTIN, then an exception is thrown in the ingress computer graphical user interface. This mispackaging may be reported to the suture manufacturer and/or to the US Food and Drug Administration as appropriate. After a satisfactory ingress, the surgical team may use the needles. At the conclusion of the procedure, the reconciliation system expects to classify and count eight needles at egress to match the ingress. If a match is identified by the software module running the reconciliation, then that suture package is considered to be reconciled.

A rudimentary surgical procedure is now discussed where only a few instruments are deployed, to illustrate exemplary displays of graphical user interface screens (e.g., ingress screens, egress screens, and reconciliation screens) that integrate a machine vision system with a graphical user interface to coordinate a process. The details of user interface screens and process flow charts may vary depending on the nature of the procedure, the human team, and the institution. Certain task assignments may be prospectively agreed upon and planned. Despite machine capabilities in machine vision and in the maintenance of instrument count tables, every step in the process may be under direct human supervision and control. That is, the example graphical user interface screens discussed herein reflect a process where machines perform tasks that machines excel at, but can always be under direct human supervision. For example, in certain embodiments, humans may be provided with the option to override machine derivations at any step in the process. In other embodiments, the system may be configured to not allow particular steps of processes described to be overridden by humans, once a step has been completed. In yet other embodiments, the system may be configured to require a new protocol to be performed, such as review by one or more supervisors, in order to modify or override a previously entered or approved step.

FIG. 8 is a screenshot showing an example graphical user interface screen displaying a set of instruments delivered by non-sterile personnel onto sterile ingress staging area 46 using a sterile technique. In accordance with an example embodiment, sterile or non-sterile personnel click the “Snap & Count” button 68 on the graphical user interface, causing an image (e.g., a photograph) to be taken of the ingress staging screen and displayed on computer display screen or monitor, such as computer 40. The machine vision system may apply pattern recognition to the objects on the screen, may apply bounding boxes 82 around them, and for each instrument, may display the type 84 and count 86 of that instrument within the computer screen. An example of a computer machine vision system that may be trained in accordance with embodiments of the invention is YOLO version 9 (https://docs.ultralytics.com/models/yolov9/).

On the same computer display screen or monitor, the graphical user interface may show separate controls and widgets for the sterile and non-sterile personnel, each working at their separate computer with separate input devices. For example, the graphical user interface may show a sterile and a non-sterile mouse icon on the same screen. The graphical user interface may also show sterile and non-sterile “Accept” buttons. The sterile and non-sterile “Accept” buttons (88b, 88a) may be correspondingly shaded on the graphical user interface display so that a third person observer upon inspecting the screen may know whether a given mouse pointer is being manipulated by the sterile or the non-sterile personnel.

If the sterile and non-sterile personnel are both satisfied that the instruments have been correctly identified and counted as indicated on the graphical user interface by the ingress bounding boxed and counted instruments, then each may place their respective mouse pointer onto the “Accept” button and click. If both click their respective “Accept” button 88a, 88b, then the instrument counts for that ingress stage are added to the reconciliation table and that ingress stage is considered closed.

If the human sterile and non-sterile personnel do not both agree, then an exception condition may be invoked, and may be reconciled and closed before proceeding. Options for closing the exception condition can depend on the reason provided by personnel for choosing not to click the respective “Accept” button. Examples of exceptions and procedures for handling such scenarios are further discussed below.

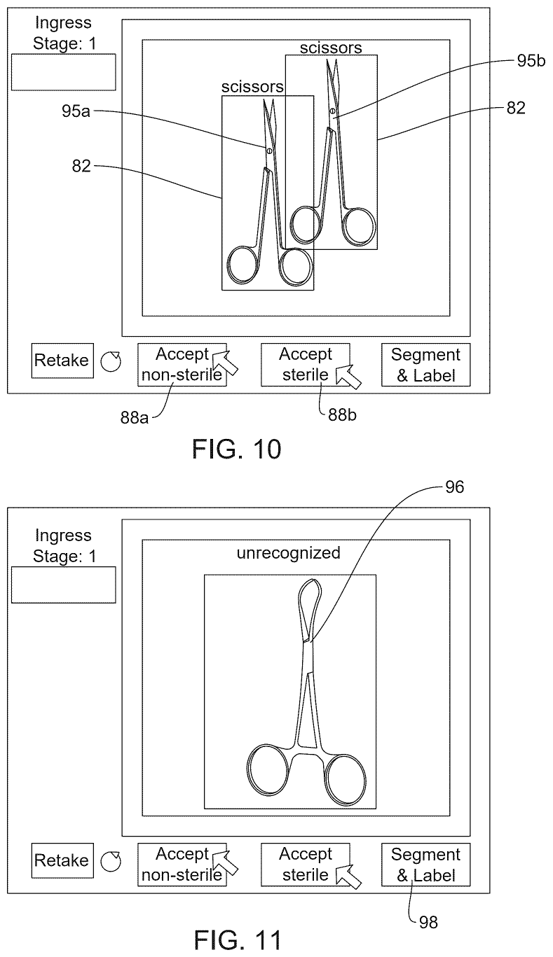

Referring to FIG. 9, a screenshot of an example graphical user interface screen for an example ingress stage is shown. This example shows one potential reason for instrument non-classification by the machine vision system: two instruments overlaid on one another such that the machine vision system cannot separately identify, classify, and count them. In some embodiments, this condition may be signaled in the graphical user interface by an “x” 90 through bounding box 82 associated with the unrecognized instrument 92. The personnel may then choose the option to rearrange the instruments so that they do not overlap onscreen, and then click a “Retake” button 94. If this allows the machine vision system to identify and classify the separate instruments 95a, 95b, as illustrated in FIG. 10, then the sterile and non-sterile personnel may click their respective “Accept” button (88a, 88b) to close the ingress stage.

Another exception mode may be if an instrument is unknown to the machine vision catalog, in which case it is not correctly recognized or labeled. FIG. 11 shows an example of an uncatalogued instrument 96, in this example, a towel clamp, that is either not in the machine vision catalog or is not recognized accurately by machine vision due to insufficient example data. This scenario may occur more frequently during an early phase of implementation (e.g., before all surgical instruments, or data associated therewith, have been loaded into the machine vision catalog). In such a scenario, a segment and label button 98 may be clicked by a user. In certain embodiments, clicking the label button 98 may cause a separate set of computer user interface tools, not illustrated here, to be displayed, allowing the use of a paintbrush-style graphical user interface to draw an instrument on the screen and then use a pop-up menu system to give it the appropriate name in the catalog.

In certain embodiments, clicking the segment and label button 98 merely produces a paintbrush icon for mouse control. In other embodiments, clicking label button 98 may activate an automated segmentation system such as SAM (Segment Anything Model https://github.com/facebookresearch/segment-anything) to assist with segmentation of the as-yet machine unidentified object. This segmentation and catalog name (SKU or Machine Vision Ontology term) as entered by the user may then be saved in the reconciliation log for this procedure, and separately for improved training of the machine vision system. If that option remains unsatisfactory, then the system may operate in a full reversion mode whereby human personnel manually count and log all instruments in the ingress stage. In this reversion mode, the graphical user interface may offer a tabular widget arrangement for personnel to manually enter instrument names and counts for a given ingress (or egress) stage.

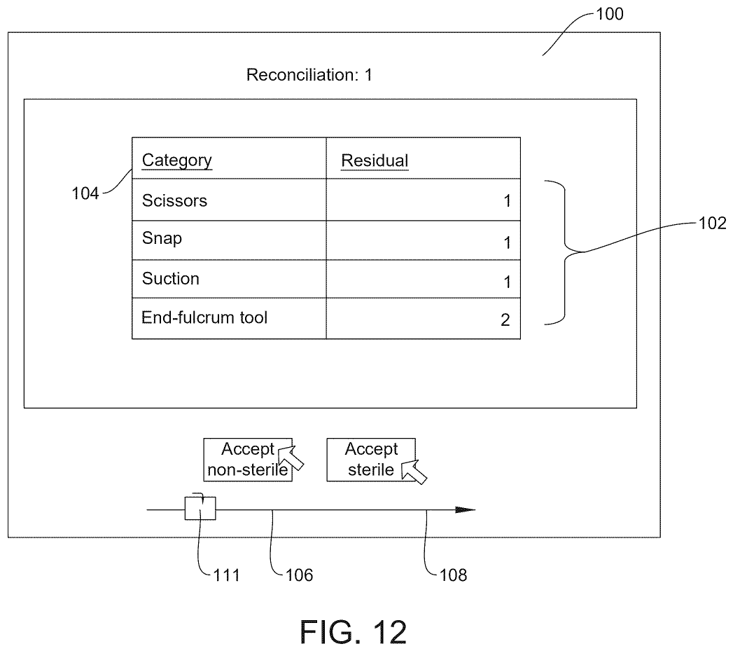

Upon completion and acceptance of the tasks in the ingress staging screen, the total reconciliation count, meaning the total count of each instrument still in the sterile field, calculated as the ingress count minus the egress count across all stages, may be updated in a stage one reconciliation screen 100 as shown in FIG. 12. Reconciliation screen 100, shown in the example graphical user interface of FIG. 12, includes a tabulation 102 of running instrument counts passed into sterile field 34 via ingress staging field 46, in accordance with an embodiment of the invention.

In this illustration, one ingress and no egress stages have occurred, but generally, the reconciliation count may be updated after acceptance of each ingress and egress stage. Whereas ingress Stage 1 screen had instrument counts associated with the instrument name at each bounding box, in the Reconciliation Stage 1 screen, there a reconciliation stage 1 table 104 of the names of all instruments and their net counts in this sterile field. In certain embodiments, the instrument names may be adapted from those specified in the Machine Vision Ontology. In certain embodiments, a reconciliation stage 1 timeline 106 may be displayed at the bottom of the screen where ingress and egress icons (e.g., photographs of imaged instruments at the different times) are shown. For example, a slider bar 108 and slider 111 may be displayed, allowing a user to slide slider 111 to the left and right along slider bar 108 to review different reconciliation stages at different times, and/or associated images captured of the surgical instruments. Other user manipulatable icons may be used. Either the sterile or non-sterile user may click on any such icons and retrieve photographs for retrospective inspection. Staging screens are preferably numbered subsequently irrespective of whether a stage is of type ingress or egress. This simplifies the display of the staging screen photographs in the timeline. In reconciliation table 104, residual instrument counts or tabulations, meaning those still in the surgical field, may be shown using a specific font (e.g., a red font) for emphasis.

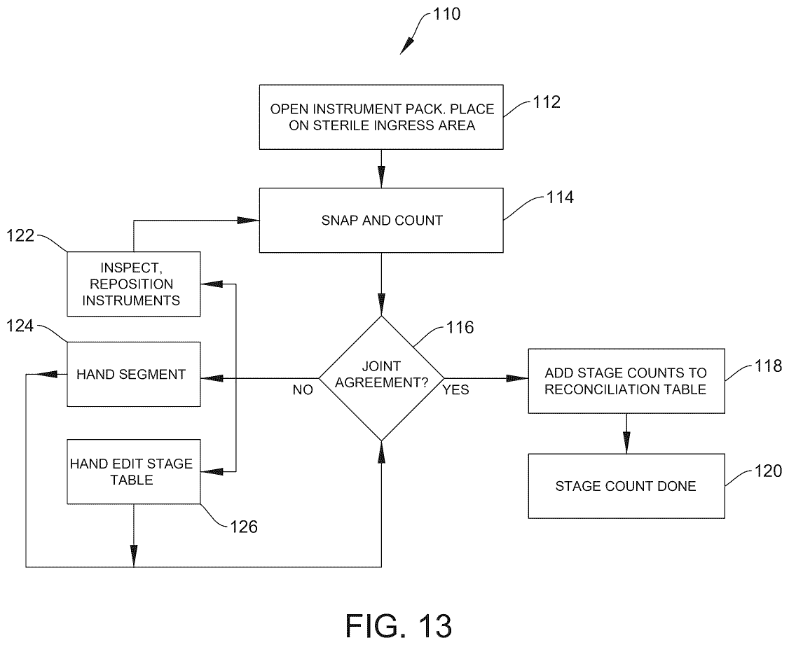

Referring to FIG. 13, disclosed is a process 110 illustrating an instrument counting method in the ingress stage according to an example embodiment of the invention. At Step 112, a person outside the sterile field (e.g., a “nonsterile person”) opens an instrument pack and places the instruments in sterile ingress staging area or field 46 (FIG. 3). At Step 114, the nonsterile person and a person inside the sterile field (e.g., a “sterile person”) causes the machine vision system to obtain an image of the instruments in sterile ingress staging area 46, via ingress camera 48, and to count the instruments based on the image (“snap and count”). For example, instruments in the image may be recognized according to the techniques described herein, and the recognized instruments may be classified as per the Machine Vision Ontology described herein. In an example embodiment, sterile and non-sterile graphical user interfaces (GUIs) may be used by the sterile person and the nonsterile person, respectively, to cause the system to snap an image and count the instruments. For example, the snap and count process may be initiated when both the sterile and nonsterile persons have selected an appropriate button in the GUI to initiate the snap and count process.

At Step 116, the count is displayed to the sterile person and the nonsterile person, who may individually confirm or dispute the count (e.g., via inputs to their respective GUIs). For example, the sterile and nonsterile GUIs may display reconciliation screen 100 (FIG. 12) to the sterile and nonsterile persons, respectively, and the sterile and nonsterile persons may select the appropriate button on the screen. Based on the input(s) from the sterile and nonsterile persons, the system may make a determination as to whether or not the sterile person and the nonsterile person both agree with the count arrived at by the system (“joint agreement?”).

At Step 118, if a determination is made that the sterile person and the nonsterile person both agree with the count (‘Yes’ at Step 116), then the counts are added to the reconciliation table, and the ingress stage count process terminates (Step 120).

If a determination is made that one or both of the sterile person and the nonsterile person do not agree with the count (‘No’ at Step 116), then the sterile person and nonsterile person have several options. For example, the sterile and nonsterile persons may decide to individually perform hand counts of the instruments (“Hand segment”) Step 124), or the sterile and nonsterile persons may decide to individually edit the annotated image generated by the system (“Hand edit stage table”) at Step 126. If hand segmenting or hand editing is selected, then the results are inputted to the system, and the method returns to Step 116 to determine whether there is now joint agreement as to the count, and repeats until the ingress stage count process proceeds to Step 118 (if joint agreement is reached—‘Yes’ at Step 116) and terminates (Step 120). In certain embodiments, if no joint agreement is reached (‘No’ at Step 116), then the sterile and nonsterile persons may inspect and reposition the instruments (Step 122) in ingress staging area 46 to enable the system to acquire a better image for analysis. Once the instruments are repositioned, process 110 returns to Step 114 (“snap and count”), and repeats until the ingress stage count process terminates Step 120).

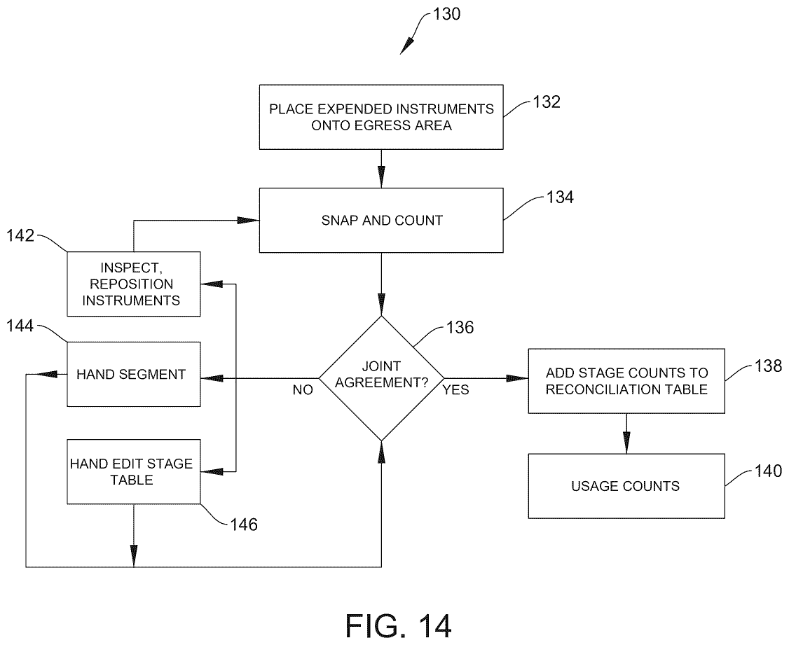

Referring to FIG. 14, disclosed is a process 130 illustrating an instrument counting method in the egress stage according to an example embodiment of the invention. At Step 132, one or more of sterile personnel place instruments to be removed into egress staging area 54 (FIG. 3). At Step 134, a nonsterile person and a sterile person inside sterile field 34 cause the machine vision system, via egress camera 56, to obtain an image of the instruments in sterile egress staging area 54, and to recognize and count the instruments being removed from sterile field 34 based on the image (“snap and count”). The snap and count process may be the same or similar to that described with respect to Step 114 of process 110. The recognized instruments may be classified as per the Machine Vision Ontology.

At Step 136, a joint agreement process may occur which is similar to Step 116 of process 110. The count is displayed to the sterile person and the nonsterile person, who may individually confirm or dispute the count (e.g., via inputs to their respective GUIs). For example, the sterile and nonsterile GUIs may display a reconciliation screen similar to reconciliation screen 100 (FIG. 12) to the sterile and nonsterile persons, respectively, and the sterile and nonsterile persons may select the appropriate button on the screen. Based on the input(s) from the sterile and nonsterile persons, the system may make a determination as to whether or not the sterile person and the nonsterile person both agree with the count arrived at by the system.

At Step 138, if a determination is made that the sterile person and the nonsterile person both agree with the count (‘Yes’ at Step 136), then counts are added to the reconciliation table (Step 138), and the egress stage count process terminates with a final stage count or performs a usage count (Step 140).

If a determination is made that one or both of the sterile person and the nonsterile person do not agree with the count (‘No’ at Step 136), then the sterile person and nonsterile person have several options, similar to those described above with respect to process 110. The sterile and nonsterile persons may decide to individually perform hand counts of the instruments (Step 144), or the sterile and nonsterile persons may decide to individually edit the annotated image generated by the system (Step 146).

If hand segmenting or hand editing is selected, then the results are inputted to the system, and the process returns to Step 136 to determine whether there is now joint agreement as to the count, and repeats until the egress stage count process proceeds through Steps 138 and 140 (if now ‘Yes’ at Step 136) and terminates or performs a usage count (Step 140). In certain embodiments, if no joint agreement is reached (‘No’ at Step 136), then the sterile and nonsterile persons may inspect and reposition the instruments (Step 142) in egress staging area 54 to enable the system to acquire a better image for analysis. Once the instruments are repositioned, process 130 returns to Step 134 (“snap and count”), and repeats until the egress stage count process terminates or does a usage count Step 140).

During process 130, an egress sub-stage analysis (further described below with respect to FIG. 18) may also occur where a separate counting occurs of those instruments used versus not used. A predictive model may be generated for a future scheduled surgical procedure, (e.g., predicting which instruments are likely to be used). The generated model may be used to generate more accurate and efficient pick lists.

In certain embodiments, the primary egress staging may call for cooperation between trained human personnel and a computer system hosting a graphical user interface (“GUI”) that supports the process and maintains internal ingress and egress instrument counts and their reconciliation. The egress process 130 has similarities to ingress process 110, such as possible requirements for agreement by personnel on machine instrument recognition, but also has some important differences. Some instruments may be egressed and counted from surgical field 34 during the surgical procedure while sterility is being maintained. Examples of this are sharp one-time use objects such as needles that came supplied with suture threads. Other instruments may not be counted through egress until the procedure is completed and the sterile field taken down. In such circumstances, the non-sterile personnel or the immediately previous sterile personnel may perform the egress process equivalently. In certain embodiments, implantable instruments may be logged in their own area of the electronic medical record to provide an accounting of which implantable instruments have been placed in which patients. For example, such instruments to be implanted in the patient may be initially logged, and if counted as part of the count (e.g., the count of all instruments, implantable and nonimplantable), then the counts for the implantable instruments may be reconciled immediately after implantation and/or at the end of a surgical procedure. In other embodiments, such implantable instruments may be given a count of zero, or logged separately under their own count procedure. In other words, the implantable instruments may be considered part of the initial plurality of surgical instruments which enter the sterile field, or separately accounted for in a separate count.

The primary egress stage counts should be completed at least before the patient emerges from anesthesia so there can be a chance to search in a body cavity for one or more instrument(s) that fail reconciliation counts. As with the ingress counting, there may be a reversion mode where traditional hand counting is employed if there is not unanimous endorsement of the machine counting by the responsible personnel. The recourse may even include an x-ray to look for a metallic instrument before emergence from anesthesia. Egress sub-stage counting based on instruments used versus not-used may be performed at a deliberate pace after the wound is closed and the patient starts emerging from anesthesia. The machine counting may be as per the Machine Vision Ontology. The counts may be subsequently mapped to an SKU system, and then employed to generate predictive instrument model usage. As noted, process 130 may include an egress sub-stage where separate counts are made of egressed instruments used versus not used during the procedure. This egress sub-stage may not be time-sensitive in the manner of the primary egress stage counting. For example, it may occur subsequent to the primary egress staging.

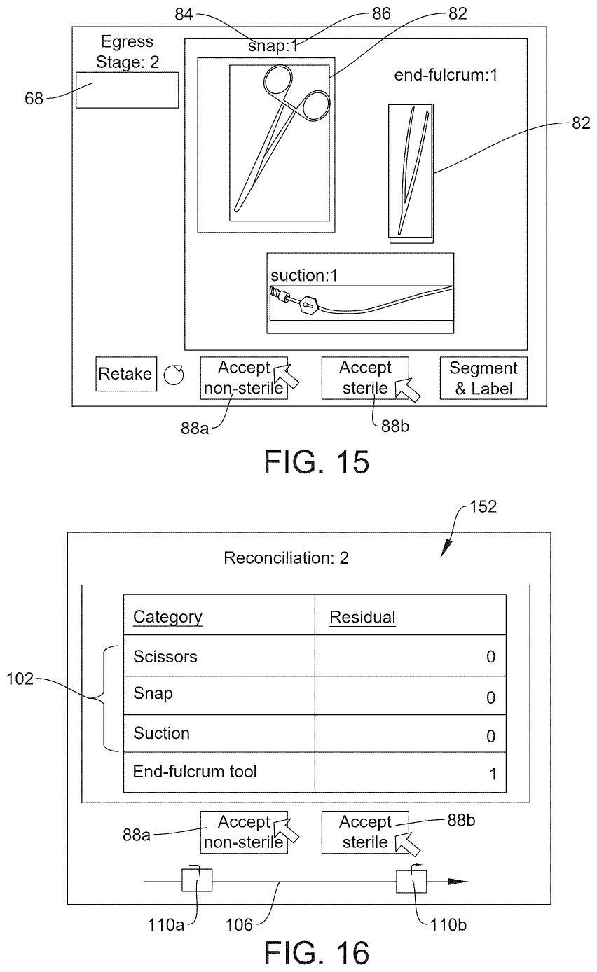

Referring to FIG. 15, a primary egress staging screen 150 of a graphical user interface is shown, similar to the ingress staging screen of FIG. 10, according to an example embodiment of the invention. In this example, instruments have been placed onto egress staging area 54 (FIG. 3). Snap and count button 68 may be clicked by a user to trigger a photograph, followed by machine identification and counting as described above. Bounding boxes 82 may be similarly placed around the instruments next to the type 84 and count 86, which may similarly be displayed for each instrument in the egress stage. If both personnel agree with the classification and counting, then they may click their respective “Accept” buttons 88a, 88b. As with ingress processing, exception modes are available for selection up to and including full reversion to manual counting.

Referring to FIG. 16, an example reconciliation screen 152 of a graphical user interface is shown, with a net count table 102 after the previous example egress stage 2 is completed. Those instruments where the egress count matches the ingress count may be set to a residual count of zero in the displayed table 102. Those instruments that have outstanding counts still in the surgical field may be listed with their outstanding counts in a red font. In this particular example, as shown, an unaccounted-for “End-fulcrum tool,” in the parlance of the Machine Vision Ontology, remains. In certain embodiments, count 102 for the tool remaining in the surgical field may be highlighted in red or some other color to indicate that it is still in the surgical field. Other notations or notifications may be utilized, such as alert, a flashing icon, an audible sound outputted from a speaker, etc. If the sterile and non-sterile personnel are satisfied with the accuracy of the reconciliation stage, they may both click their respective “Accept” buttons 88a, 88b and proceed.

At the conclusion of the procedure, when all ingress and egress counts have been performed and jointly approved, the net instrument counts in reconciliation screen 152 should all be zero. In certain embodiments, at the bottom of the screen, a timeline 106 may similarly be displayed where the photographs for ingress and egress stages are shown in icon form. These icons may be annotated with an arrow oriented to indicate whether a particular icon points to an ingress stage photograph or an egress stage photograph. Slider bar 108 and sliders 111a, 111b may similarly be used to show the times both of ingress and egress of an instrument. Slider 111a may show an ingress time of a surgical instrument and slider 111b may show an egress time of a surgical instrument.

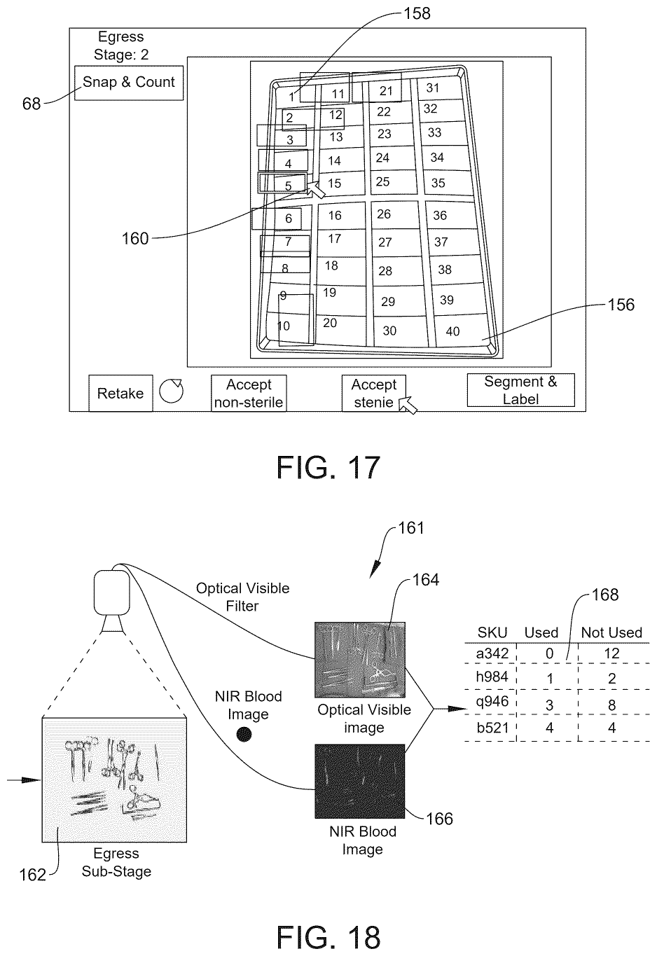

Needle counts are an important part of egress counting for the particular risk retained needles pose to the safety of the patient and to personnel. Referring to FIG. 17, a screenshot 154 of a graphical user interface illustrating application of the Machine Vision Ontology to egress counting of suture needles is shown. At ingress, suture packs equipped with curved needles of different sizes attached to suture threads of different types may be supplied onto the surgical field via ingress staging area 46.

Aspects of example suture packages 76 are shown in FIG. 7. As sutures are used during a surgical procedure, sterile personnel may place the used curved needles onto a foam suture needle deposit 156. This serves two purposes. The first is to bury the sharp tip away from working human personnel. The other is to offer a holding area for the subsequent joint counting of the curved needles by the sterile and non-sterile personnel. When a person clicks the Snap & Count button 68 in screen 154, a photo is taken of egress field 54 via camera 56. The curved needles may be recognized by machine vision and each identified with a curved suture needle bounding box 158.

In certain embodiments, when a user holds his or her curser over foam suture needle deposit 156, which is in egress staging area 54, the machine vision system may locate and count the number of needles in foam suture needle deposit 156, compare this count with the number of needles in egress staging area 54, and brighten a bounding box in the graphical user interface of screen 154, within foam suture needle deposit 156, as a source of emphasis. To enhance the human capability of inspection, if a mouse icon is dragged over a bounding box, then it may shown as a highlighted curved suture needle bounding box 160. This may provide a way for humans to inspect the bounding box counts without manipulating the sharp objects for a retake while assuring that there are no free needles inadvertently left in the patient's body. An operator may, for example, systematically click on each of boxes ‘1’ to ‘40’ in foam suture needle deposit 156 to highlight each box and count the total number of suture needles.

Where the affixed curved needles may be individually counted by the machine vision system, the required visual information to distinguish the origin of one curved needle from the other may not be present. The hierarchical Machine Vision Ontology offers a hierarchical term under “curved suture needle” where the total egress needle count may be attached. If this count matches the total ingress count, then the surgical team may safely conclude that no needles of any type are left in the patient's body. The recognition of needles against a Machine Vision Ontology ensures that patient safety is maintained even when one curved needle type is misrecognized as another due to similarity of shape and size.

Referring to FIG. 18, a diagram of an example embodiment of an egress sub-stage counting configuration 161 is shown, where instruments used versus not-used are separately classified and counted. After primary egress staging and reconciliation (which has priority due to time-sensitivity and patient safety), the reconciled instruments may be placed on a separate machine vision egress sub-staging zone 162. Sub-stage counting configuration 161 may be a fully or at least partially automated idempotency system for sub-stage classification and counting.

In an automated embodiment, egress sub-stage camera (e.g., camera 56) may have both a visible wavelength lens and an infrared lens that captures a visible wavelength optical image 164 and an infrared image 166, respectively. The images may be electronically transferred to a computer for machine vision classification and counting of the instruments. For example, the optical visible filter shown in FIG. 18 can be egress camera 56 with and/or without an optical filter adapted to detect hemoglobin, other body fluid, or body tissue, or a third imaging device equipped with these features. In certain embodiments, ingress camera 48 may also be equipped with these features. In a subsequent software process, the infrared image 166 may be overlaid onto the visible wavelength optical image 164. Those instruments with an infrared signal that superimpose over an instrument according to a statistical detection threshold may be counted as having blood on them, and therefore, counted as having been used. The column for each SKU in an instrument usage log 168 may be incremented for each SKU instrument assessed to have been used according to a statistical analysis of the infrared signal in the infrared image 166 that corresponds spatially to an instrument identified on the visible wavelength optical image 164.

In a comparatively less automated embodiment, human personnel may separate the used instruments onto egress sub-stage zones, one for instruments used and one for instruments not used. These may be separately photographed, and the images may be subjected to machine vision instrument classification and counting.

Other embodiments to collect data on which instruments were used and not used may be utilized. For example, the instruments could be etched with a barcode matching the SKU. At the end of the procedure, those instruments which were not used may be placed under the barcode reader and counted. Those instruments that were used may be placed under the barcode reader to allow the computer to identify and store information about these used instruments. This information could be achieved, for example, via two separate barcode readers, one which reads the barcodes on instruments used and one which reads the barcodes on instruments not used. The system may be configured to display a widget in a graphical user interface where a human technician can indicate those instruments used versus not used. In other embodiments, the SKU deployment and usage can be captured using, for example, radiofrequency identifier devices and manually edited spreadsheets. Regardless of the method of counting, counting used versus non used instruments is done in order to generate a predictive model of instrument usage for future procedures.

One of ordinary skill in the art will appreciate that the systems and methods described herein may be extended to address other circumstances not described, such as, for example, a situation where an instrument is dropped inadvertently onto the floor. For such instances, a correction box labeled “Dropped” may be utilized, and the objects in it may be counted at the last egress stage.

Various computer vision software packages may be used for accurate object classification. One example of a suitable computer vision software is YOLO version 9 (https://docs.ultralytics.com/models/yolov9/). As noted previously, the egress sub-stage counting may enable the generation of a statistical predictive model of utilization. The predictive utilization process may involve the capture and incorporation of additional data available in advance of the procedure. A statistical model may be generated connecting the predictive data to the data gathered by the first stage with a machine vision inspection and analysis of instruments supplied to the sterile table and those used.

When a surgical procedure is scheduled, there may be associated information that may be used productively. The predictive utilization process may leverage this information in addition to the machine vision classification and counting of the egress instruments. For example, U.S. Pat. No. 10,672,506 to Butler, filed on Apr. 29, 2013 and titled ‘Method and device for generating a graphical user interface for procedure-based medical charge capture’ (Hereafter, ‘Butler’), which is hereby incorporated by reference herein in its entirety, teaches how to use surgical information predictively to place diagnosis and billing codes efficiently on a graphical user interface. Present methods may use information such as diagnosis and service codes proactively to provide an estimate of the likely usage of surgical instruments during a surgical procedure.

When a medical service is provided by a healthcare provider, to compose a bill the diagnosis and service must be described (“coded”) with accepted diagnosis and services codes. A common system of diagnosis codes in current use is the International Classification of Disease, version 10 (ICD-10), provided by the World Health Organization. In addition, a common system of service codes is the Common Procedural Terminology (CPT), provided by the American Medical Association. Commonly, when a surgical procedure is scheduled, both ICD-10 and CPT codes are furnished to the operating room scheduling personnel for a given procedure. This information may be used to predict the length of the surgical procedure, the instruments kit required, personnel needed, and the time that a given operating room will be used to carry out the procedure safely, effectively, and efficiently. Some operating room administrative arrangements have custom surgical scheduling codes that they employ instead of ICD-10 and CPT codes for scheduling purposes. Those codes may be used instead of ICD-10 and CPT codes in this intervention.

In certain embodiments, ordinal logistic regression may be selected because it has favorable statistical properties. The SKU for a commonly used instrument, such as Metzenbaum scissors, is M236. In the ordinal logistic regression, this SKU may be associated with a usage index ranging, for example, from 0 to 10. Each value in this range may correspond to the predicted number of Metzenbaum scissors to be employed in that procedure. The ordinal logistic regression may produce a probability value associated with the interval ranging from 0 to 10, given the estimated probability of the number of Metzenbaum scissors being used.

In another embodiment, a large language model (LLM) may be used to predict the number of instruments used in a surgical procedure. This has the advantage of operating on a large corpus of medical and surgical data pertaining to the medical institution, the patient, the surgeon, the anesthetist, and other considerations to be elucidated by further study.

In a multivariate logistic regression, this information may be yielded for the entire range of surgical instruments, whether disposable or not, to be associated with a prediction. The availability of this predicted utilization may then be employed to close the loop to advance the safety, efficacy, and efficiency of the planned surgical procedure.

The predictive model may enable the generation of a complete and efficient pick list for a future scheduled sterile procedure, the description of which may focus on the tangible resources, including both disposable and non-disposable items, to be laid out on an instrument table. The model may also be employed to predict other properties, such as operating room time requirements, and hours to be spent by various personnel. Items to be predicted may be tokenized, assigned an embedding, then added to a large language model for training. An example large language model suitable for this purpose is the Databricks LLM (https://www.databricks.com/resources/ebook/tap-full-potential-llm).

In certain embodiments, a pick-list may be guided according to the statistical model of predicted utilization. An operating room supply organization may employ a fine grain or a coarse grain strategy for preparing packs. In a coarse grain strategy, surgery scenarios are broadly defined so that a single pack is likely to serve many surgical procedures, patients, surgery types, and surgeons. In certain embodiments, a cluster analysis may be performed on the elaboration of predictive variables and utilization variables using cluster analysis as taught by Butler. The utilization variables in each cluster are then prepared and sterilized in a pack.

In a fine grain delivery strategy, the surgeon's name, the operating room, the institution, the patient identifier, and the predicted ICD-10 and CPT codes are supplied to the predictive model, which may then generate a predicted utilization. In a fine grain strategy, the number of instruments and the predicted utilization may be likely to be smaller. This smaller pack may be then supplied to the operating room on a timely basis for the scheduled surgical procedure. Smaller packs would require less time for counting thus saving personnel and operating room time. The customization of the delivery strategy may be extended to a just-in-time routine. The predicted utilization may be applied to the operating room supply personnel with the advance notice needed to secure the predicted supplies, place them in a pack, sterilize it, and deliver it to the operating room. In a fine grain delivery strategy, a utilization pack may be likely to have a smaller number of supplies. This may enable it to be prepared more expeditiously. A smaller number of supplies translates into efficiency by reducing the number of disposable and non-disposable supplies needed to be purchased, stocked, and, in the case of disposable supplies, discarded in biohazard channels, or, in the case of non-disposable supplies, restocked and then re-sterilized for the next surgical case to prepare for its predicted use.

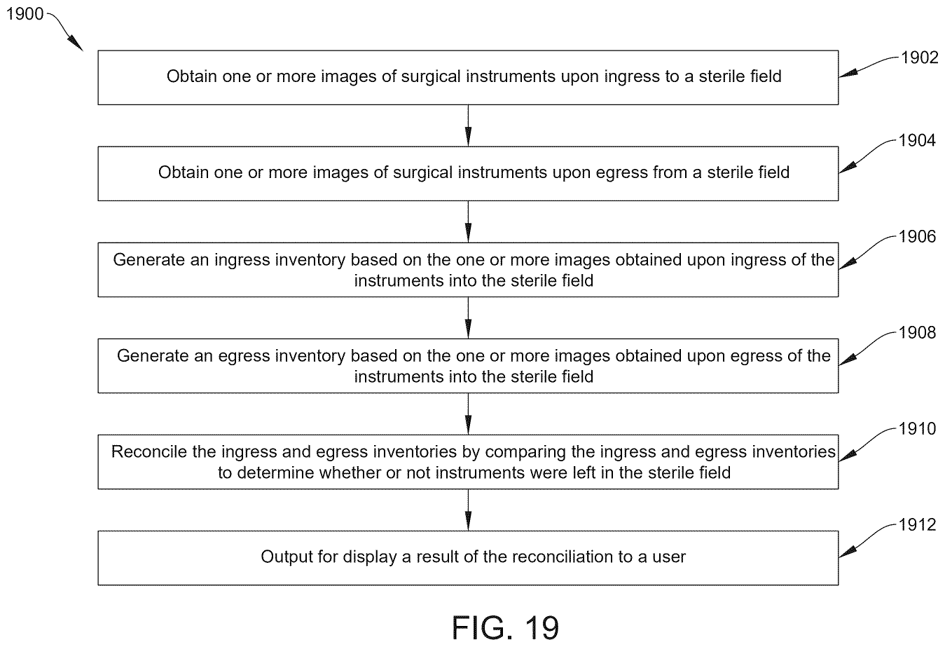

Referring now to FIG. 19, a flow chart of a method 1900 according to an example embodiment is shown. At Step 1902, one or more images of the surgical instruments are obtained as the instruments ingress into the sterile field. The image may be collected by a single imaging device or by more than one imaging device. The one or more imaging devices may include any suitable device, including but not limited to a camera, a barcode reader, an RFID reader, or a combination of two or more of the foregoing devices. The image obtained by the one or more imaging devices may be stored in temporary memory or storage or may be filed in permanent memory for later use.

At Step 1904, a corresponding image is taken of the surgical instruments as they egress from the sterile field. Again, the image may be collected by a single imaging device or by more than one imaging device, and the imaging devices may include any suitable device, including but not limited to a camera, a barcode reader, an RFID reader, or a combination of two or more of the foregoing devices. The one or more images obtained by the one or more imaging devices may be stored in temporary memory or storage or may be filed in permanent memory for later use.

At Step 1906, an ingress inventory is generated based upon the images from the ingress station. The ingress inventory may consist of at least a listing of each item which entered the ingress station. Each item may be annotated on the inventory by its SKU. The use of SKU has an equivalent machine vision ontology string vector describing the instrument. Each inventory may be conducted in real-time or in batch mode. There may be several distinct ingress events, each having a separate inventory. These separate inventories may then be aggregated together to form an overall ingress inventory.

At Step 1908, an egress inventory is generated at based upon the equivalent images taken of the egress station. This inventory consists of at least a listing of each item which entered the egress station. It will be appreciated that by the end of the surgical procedure, a set of the surgical instruments will have been egressed from the sterile field. If all of the surgical instruments that entered the sterile field are egressed from it, then the set of surgical instruments egressed from it will equal the plurality of instruments which entered the sterile field, unless implantable surgical instruments are left in the patient. Implantable surgical instruments can be accounted for via a separate accounting as discussed herein. If any surgical instruments, implantable or otherwise) are unintentionally left in the surgical field (e.g., in the patient or outside of the patient but in the sterile field), then the set of surgical instruments egressed from the sterile field will not match or equal the plurality of surgical instruments which entered it. Each item may be annotated on the inventory by its SKU. The use of SKU has an equivalent machine vision ontology string vector describing the instrument. Each inventory may be conducted in real-time or in batch mode. There may be several distinct egress events, each having a separate inventory. These separate inventories may then be aggregated together to form an overall egress inventory.

At Step 1910, the ingress inventory and the egress inventory are reconciled to determine if any instruments are left in the sterile field. The reconciliation between the ingress and egress inventory may be conducted in real-time or in batch mode. Each reconciliation can be first netted across the various ingress inventories and then across all the various egress inventories. Further, the inventories may be reconciled in a real-time form, such that a listing of all instruments remaining in the sterile field is presented to at least one user.

At Step 1912, the output of the reconciliation is displayed to one or more users. The display to the users may require that a single user acknowledge the reconciliation. The display may further require that at least one sterile and one non-sterile user acknowledge the reconciliation. Further, the display may sound an audio alarm as well as a visual alarm when the reconciliation indicates that there may be an instrument left in the sterile field after the procedure has been deemed complete.

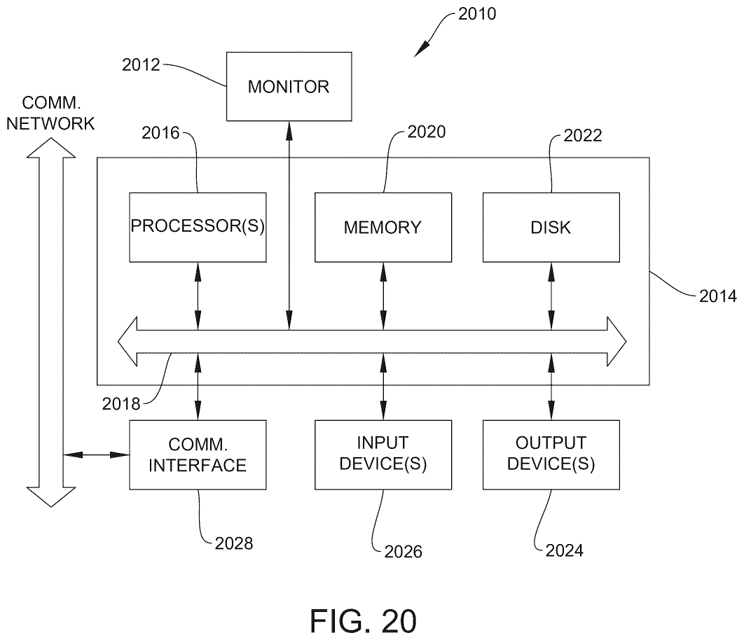

Referring now to FIG. 20, a block diagram of an example system 2010 is illustrated for a machine vision instrument reconciliation, imaging system, and/or prediction system as described herein. System 2010 may be local to or remote from the surgical procedure. In one example, at least some of the functionality performed by system 2010 may be offered as a Software-as-a-Service (SaaS) option. SaaS refers to a software application that is stored in one or more remote servers (e.g., in the cloud) and provides one or more services (e.g., surgical instrument reconciliation) to remote users.

In one embodiment, system 2010 includes a monitor or display device 2012, a computer system 2014 (which includes processor(s) 2016, bus subsystem 2018, memory subsystem 2020, and disk subsystem 2022), user output devices 2024, user input devices 2026, and communications interface 2028. Monitor 2012 can include hardware and/or software elements configured to generate visual representations or displays of information as described herein. Some examples of monitor 2012 may include familiar display devices, such as a television monitor, a cathode ray tube (CRT), a liquid crystal display (LCD), a light-emitting diode (LED) display, or the like. In some embodiments, monitor 2012 may also provide an input interface, such as incorporating touch screen technologies.

Computer system 2014 can include familiar computer components, such as one or more central processing units (CPUs), memory or storage devices, graphics processing units (GPUs), communication systems, interface cards, or the like. As shown in FIG. 20, computer system 2014 may include at least one hardware processor 2016 that communicates with a number of peripheral devices via bus subsystem 2018. Processor(s) 2016 may include commercially available central processing units or the like. Bus subsystem 2018 can include mechanisms for letting the various components and subsystems of computer system 2014 communicate with each other as intended. Although bus subsystem 2018 is shown schematically as a single bus, alternative embodiments of the bus subsystem may utilize multiple bus subsystems. Peripheral devices that communicate with processor(s) 2016 may include memory subsystem 2020, disk subsystem 2022, user output devices 2024, user input devices 2026, communications interface 2028, or the like.

Processor(s) 2016 may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform the various functions and steps of the embodiments and methodologies described herein, such as by executing instructions stored in memory subsystem 2020 and/or disk subsystem 2022 or another computer program product.

Memory subsystem 2020 and disk subsystem 2022 are examples of physical storage media configured to store data, such as instructions executable by the one or more processors 2016 to perform the operations described herein. Memory subsystem 2020 may include a number of memories or memory devices including random access memory (RAM) for volatile storage of program code, instructions, and data during program execution and read only memory (ROM) in which fixed program code, instructions, and data are stored. Disk subsystem 2022 may include a number of file storage systems providing persistent (non-volatile) storage for programs and data. Other types of physical storage media include floppy disks, removable hard disks, optical storage media such as compact disc - read-only memories (CD-ROMS), digital video disc (DVDs) and bar codes, semiconductor memories such as flash memories, read-only-memories (ROMS), battery-backed volatile memories, networked storage devices, or the like. Memory subsystem 2020 and disk subsystem 2022 may be configured to store programming and data constructs that provide functionality or features of techniques discussed herein. Software code modules and/or processor instructions that when executed by processor(s) 2016 implement or otherwise provide the functionality may be stored in memory subsystem 2020 and disk subsystem 2022. Memory subsystem 2020 and/or disk subsystem 2022 may be a non-transitory computer readable storage medium.

User input devices 2026 can include hardware and/or software elements configured to receive input from a user for processing by components of system 2010. User input devices can include all possible types of devices and mechanisms for inputting information to computer system 2014. These may include a keyboard, a keypad, a touch screen, a touch interface incorporated into a display (e.g., computer display 40), audio input devices such as microphones and voice recognition systems, imaging input devices such as cameras (e.g., cameras 48, 56), barcode readers, and/or other types of input devices. In various embodiments, user input devices 2026 may also include a computer mouse, a trackball, a track pad, a joystick, a wireless remote, a drawing tablet, a voice command system, an eye tracking system, or the like. In some embodiments, user input devices 2026 are configured to allow a user to select or otherwise interact with objects, icons, text, or other displayed elements or the like within the various graphical user interfaces described herein, that may appear on monitor 2012 via a command, motions, or gestures, such as a click of a button, voice command, keystroke, or the like.

User output devices 2024 can include hardware and/or software elements configured to output information to a user from components of computer system 2010. User output devices can include all possible types of devices and mechanisms for outputting information from computer system 2014. These may include a display device (e.g., monitor 2012) such as, for example, computer display 40 and/or reconciliation screen 52 of FIG. 3), a printer, a touch or force-feedback device, audio output devices, or the like.

Communications interface 2028 can include hardware and/or software elements configured to provide unidirectional or bidirectional communication with other devices. For example, communications interface 2028 may provide an interface between computer system 2014 and other communication networks and devices, such as via an internet connection.

Computer readable program instructions described herein can be downloaded to respective computing/processing devices from a computer readable storage medium or to an external computer or external storage device via a network, for example, the Internet, a local area network, a wide area network and/or a wireless network. The network may comprise conductive transmission cables, optical transmission fibers, wireless transmission, routers, firewalls, switches, gateway computers and/or edge servers. A network adapter card or network interface in each computing/processing device may receive computer readable program instructions from the network and forward the computer readable program instructions for storage in a computer readable storage medium within the respective computing/processing device.

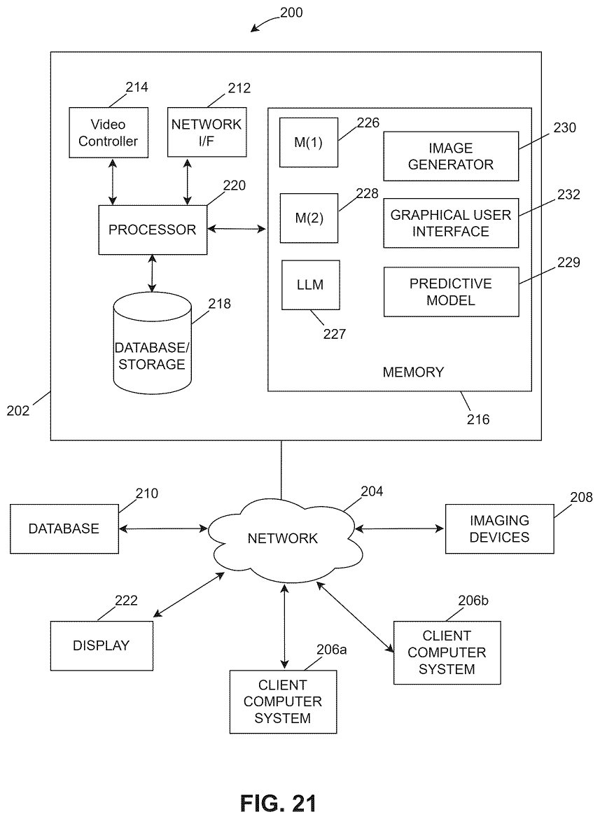

Referring to FIG. 21, an example computing environment 200 is shown for executing the functions and methodologies of the various embodiments of the invention described herein. Computing environment 200 may include a server 202 in communication with, via network 204, one or more of a user or client computer system 206a, 206b, one or more imaging devices 208, a database 210, and one or more displays 222. Server 202 and client computer systems 206a, 206b may each include computer system/information processing device 2010, described above with respect to FIG. 20, certain components thereof, or a different computer system.

Client computer systems 206a, 206b may respectively include, for example, sterile ingress computer/display 40 and egress computer/display 50, and each of these may include a laptop computer, a tablet computer, a netbook computer, a personal computer (PC), a desktop computer, a personal digital assistant (PDA), a smart phone, a thin client, or any programmable electronic device capable of executing computer readable program instructions.

Imaging devices 208 may include ingress and egress cameras 48, 56 described above with respect to FIG. 3, one or more components thereof, and/or a different imaging device. Database 210 may be remotely configured relative to server 202 of computing environment 200, and in communication therewith via network 204. Display 222 can include display 52, and be in operative communication with video controller 214 via network I/F 212 for displaying images on reconciliation screen 52 for all personnel to monitor.

Network 204 may include a local area network (LAN), a wide area network (WAN) such as the Internet, or a combination of the two, and include wired, wireless, or fiber optic connections. In general, network 204 can be any combination of connections and protocols known in the art that will support communications between server 202 and client computer systems 206a, 206b, imaging devices 208, database 210, and display 222 via their respective network interfaces.

It will be understood that the functional division among components of computing environment 200 are not to be construed as a limiting example. In certain embodiments, system server 202 of computing environment 200 may include a network interface (I/F) 212, a video controller 214, memory 216, one or more databases/storage 218, and at least one processor 220 in communication with network interface (I/F) 212, video controller 214, memory 216, and database 218.

In certain embodiments, memory 216 comprises a non-transitory computer readable medium that stores instructions executable by the processor 220 to perform the functions and methodologies of the various embodiments of the invention described herein. For example, memory 216 may store a first machine learning model (M1) 226, a second machine learning model (M2) 228, a large language model (LLM) 227, a predictive model 229, an image generator module 230 for processing and adjusting image data to produce annotated images in accordance with outputs from first and second machine learning models 226, 228 based on outputs from imaging devices 208 (e.g., cameras 48, 56) and inputs from client computer systems 206a, 206b (e.g., sterile ingress computer/display 40 and/or egress computer/display 50), and a graphical user interface module 232 configured to communicate one or more graphical user interfaces, as described herein, to and for display on client computer systems 206a, 206b and/or display 222 via video controller 214. In certain embodiments, client computer systems 206a, 206b may be integrated into a single client computer system in communication with server 202 via network 204. Graphical user interface module 232 may be configured to, for example, provide the various graphical user interfaces described above with respect to FIGS. 4, 8-12, and 15-17.

First machine learning model M1 226 may be a deep learning neural network model trained and optimized for high specificity to surgical instruments, and second machine learning model 228 may be a deep learning neural network model which is also trained and optimized for high specificity to surgical instruments. For example, first machine learning model 226 may be trained to identify surgical instruments from image data inputted from ingress camera 48, and second machine learning model 228 may be trained to identify surgical instruments from image data inputted from egress camera 54. First and/or second machine learning models 226, 228 may be trained to distinguish between visually similar instruments based on the presence or absence of a decorative discriminator applied to one or more of the instruments as described herein. Alternatively, in certain embodiments, one machine learning model may be utilized which is trained to identify surgical instruments from image data inputted from either ingress camera 48 or egress camera 54.

In certain embodiments, ingress computer 40 and/or egress computer 50 may each include some or all of the features of server 202, and operate independently. For example, ingress computer 40 (e.g., client computer system 206a) may include machine learning model M(1) 226 and egress computer 50 (e.g., client computer system 206b) may include machine learning model M(2) 228. LLM 227 may be used to generate and/or update predictive model 229 to predict surgical instruments to be used in a surgical procedure as described herein.

Each of the modules and models stored in memory 216 of server 202 may include one or more sub modules or models to perform various example functions and methodologies of the present invention, be implemented by any combination of any quantity of software and/or hardware modules or units, and reside within memory 216 of server 202 for execution by a processor, such as processor 220.

Database 218 and remote database 210 may include any non-volatile storage media known in the art. For example, databases 210, 218 can be implemented with a tape library, optical library, one or more independent hard disk drives, or multiple hard disk drives in a redundant array of independent disks (RAID). Similarly, image data in databases 210, 218 may conform to any suitable storage architecture known in the art, such as a file, a relational database, an object-oriented database, and/or one or more tables. The above described system and/or components thereof may be used to implement various methodologies of the present invention described herein, including processes 110, 130, and 1900, described above with respect to FIGS. 13, 14, and 19.

Each element in flowcharts shown or methodologies described herein, depicts a step or a group of steps of novel computer-implemented systems and methodologies for managing a plurality of instruments in a sterile field, based on images obtained from imaging devices, and outputs generated from one or more deep learning models in concert with one or more pre-set or user-adjustable parameters. Each step of the methodologies described herein may contain one or more sub-steps. For purposes of illustration and explanation, these steps, as well as all other steps identified and described, are presented in a certain logical order. However, it will be appreciated that any exemplary embodiments described herein can contain an alternate order of the steps adapted to a particular application of a technique disclosed, and that any such variations and/or modifications are intended to fall within the scope of the invention. The depiction and description of steps in any particular order is not intended to exclude embodiments having the steps in a different order, unless required by a particular application, explicitly stated, or otherwise clear from the context.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a non-transitory computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts, block diagrams, and process flows depicted in the Figures illustrate the architecture, functionality, and operation of possible example implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions and steps noted in the blocks may occur out of the order noted in the Figures, and/or without all of the steps illustrated (e.g., such processes may be performed with certain steps omitted). For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

During a surgical procedure, hundreds of sterile instruments, both disposable and non-disposable, must be accurately counted at the start and conclusion. This is especially critical for procedures involving sharp instruments, body cavities (such as the abdomen or thorax), or intraoperative MRI. Ensuring accurate counts can be laborious, inefficient, and prone to human error. One of ordinary skill in the art will appreciate that embodiments of the present invention can help ensure accurate counts in a more efficient and less labor-intensive manner.

The above-described embodiments are provided by way of example and are not intended to limit the scope of the invention. Persons of ordinary skill in the art will appreciate that various modifications and changes may be made without departing from the spirit and scope of the invention. For example, while embodiments of the invention may include identification of surgical instruments by way of barcode on the instrument and the use of a barcode reader by the user, one of ordinary skill in the art would understand that identification of surgical instruments can be made by way of an RFID tag on the instrument and the use of an RFID reader by the user. It should be also be understood that features described with respect to one embodiment may be used with other embodiments.

What is claimed is:

1. A system for managing a plurality of instruments in a sterile field, the system comprising:
at least one imaging device;
one or more processors; and
a non-transitory computer readable storage medium coupled to the one or more processors and
storing instructions thereon that, when executed by the one or more processors, cause the one or more processors to:
obtain one or more images of the plurality of instruments via the at least one imaging device upon ingress of the plurality of instruments into the sterile field;
obtain one or more images of a set of the plurality of instruments via the at least one imaging device upon egress of the set of the plurality of instruments from the sterile field;
generate an ingress inventory based on the one or more images obtained upon ingress of the plurality of instruments into the sterile field;
generate an egress inventory based on the one or more images obtained upon egress of the set of the plurality of instruments from the sterile field;
reconcile the ingress and egress inventories by comparing the ingress and egress inventories to determine whether or not one or more instruments were left in the sterile field; and
output for display a result of the reconciliation to a user.

2. The system of claim 1, wherein the at least one imaging device includes a first imaging device configured to obtain one or images of the plurality of instruments upon ingress of the plurality of instruments into the sterile field, and a second imaging device configured to obtain one or more images of the set of the plurality of instruments upon egress of the set of the plurality of instruments from the sterile field.

3. The system of claim 2, wherein the instructions, when executed by the one or more processors, further cause the one or more processors to:
identify each instrument introduced into the sterile field based on the one or more images obtained by the first imaging device upon ingress of the plurality of instruments to the sterile field; and
identify each instrument removed from the sterile field based on the one or more images obtained by the second imaging device upon egress of the set of the plurality of instruments from the sterile field.

4. The system of claim 3, wherein to identify each instrument introduced and removed from the sterile field, the instructions, when executed by the one or more processors, further cause the one or more processors to:
input the one or more images obtained by the first imaging device upon ingress to a first machine learning model trained to identify instruments from image data; and
input the one or more images obtained by the second imaging device to a second machine learning model trained to identify instruments from image data.

5. The system of claim 4, wherein at least one of the first and second machine learning models is trained to distinguish between visually similar instruments based on the presence or absence of a decorative discriminator applied to one or more of the instruments.

6. The system of claim 2, wherein the at least one imaging device further comprises a third imaging device configured to read scannable codes carried by instruments, wherein the instructions, when executed by the one or more processors, further cause the one or more processors to:
identify each instrument introduced into the sterile field based on a scannable code read by the third imaging device upon ingress or based on one or more images obtained by the first imaging device upon ingress; and
identify each instrument removed from the sterile field based on a scannable code read by the third imaging device upon egress or based on the one or more images obtained by the second imaging device upon egress.

7. The system of claim 6, wherein the instructions, when executed by the one or more processors, further cause the one or more processors to:
determine a number of instruments in a package based on a scannable code on the package read by the third imaging device upon ingress into the sterile field;
determine a number of instruments in the package based on one or more images obtained by the first imaging device; and
alert a user when the number of instruments determined by the system based on the scannable code differs from the number of instruments determined by the system based on the one or more images.

8. The system of claim 1, wherein the instructions, when executed by the one or more processors, further cause the one or more processors to prompt a user for confirmation that the instruments identified by the system upon ingress or egress are counted only once.

9. The system of claim 1, further comprising displaying, on a display screen, a graphical user interface with separate controls for sterile and non-sterile users.

10. The system of claim 9, further comprising:
a sterile ingress computer for one or more sterile users to interact with the graphical user interface; and
an egress computer for one or more non-sterile users to interact with the graphical user interface.

11. The system of claim 2,
wherein the instructions, when executed by the one or more processors, further cause the one or more processors to
obtain, using the second imaging device, images of the instruments with an optical filter adapted to detect hemoglobin, other body fluid, or body tissue; and
classify the set of instruments identified by the machine learning model during egress as used or not-used based on the presence or absence, respectively, of body fluid and tissue on the instruments.

12. The system of claim 11, wherein the instructions, when executed by the one or more processors, further cause the one or more processors to generate or update a predictive model that predicts which instruments are likely to be used in a procedure based on a classification of instruments as used or not-used.

13. The system of claim 12, wherein the predictive model comprises an ordinal logistic regression configured to provide a probability value corresponding to the number of instruments predicted to be used in a particular surgical procedure.

14. The system of claim 12, wherein the predictive model comprises a large language model trained using a data set that includes usage data based on the classification of instruments used in a particular surgical procedure.

15. The system of claim 12, wherein the predictive model comprises a multivariate logistic regression configured to predict the number of instruments used in a particular surgical procedure.

16. A method for managing a plurality of instruments in a sterile field, the method comprising:
obtaining, using at least one imaging device, one or more images of the plurality of instruments upon ingress of the plurality of instruments into the sterile field;
obtaining, using the at least one imaging device, one or more images of a set of the plurality of instruments upon egress of the set of the plurality of instruments from the sterile field;
generating, using one or more processors, an ingress inventory based on the one or more images obtained upon ingress of the plurality of instruments into the sterile field;
generating, using the one or more processors, an egress inventory based on the one or more images obtained upon egress of the set of the plurality of instruments from the sterile field;
reconciling, using the one or more processors, the ingress and egress inventories by comparing the ingress and egress inventories to determine whether or not one or more instruments were left in the sterile field; and
outputting, using a display device, a result of the reconciliation to a user.

17. The method of claim 16, wherein the at least one imaging device includes a first imaging device configured to obtain one or more images of the plurality of instruments upon ingress of the plurality of instruments into the sterile field, and a second imaging device configured to obtain one or more images of the set of the plurality of instruments upon egress of the set of the plurality of instruments from the sterile field.

18. The method of claim 16, further comprising:
identifying, using the one or more processors, each instrument introduced into the sterile field based on the one or more images obtained by the first imaging device upon ingress of the plurality of instruments to the sterile field; and
identifying, using the one or more processors, each instrument removed from the sterile field based on the one or more images obtained by the second imaging device upon egress of the set of the plurality of instruments from the sterile field.

19. The method of claim 18, wherein to identify each instrument introduced and removed from the sterile field, the one or more processors are further used to:
input the one or more images obtained by the first imaging device upon ingress to a first machine learning model trained to identify instruments from image data; and
input the one or more images obtained by the second imaging device to a second machine learning model trained to identify instruments from image data.

20. The method of claim 19, wherein at least one of the first and second machine learning models is trained to distinguish between visually similar instruments based on the presence or absence of a decorative discriminator applied to one or more of the instruments.

21. The method of claim 17, wherein the at least one imaging device further comprises a third imaging device configured to read scannable codes carried by instruments, and wherein the one or more processors are further used to:
identify each instrument introduced into the sterile field based on a scannable code read by the third imaging device upon ingress or based on one or more images obtained by the first imaging device upon ingress; and
identify each instrument removed from the sterile field based on a scannable code read by the third imaging device upon egress or based on the one or more images obtained by the second imaging device upon egress.

22. The method of claim 21, wherein the one or more processors are further used to:
determine a number of instruments in a package based on a scannable code on the package by the third imaging device upon ingress into the sterile field;
determine a number of instruments in the package based on one or more images obtained by the first imaging device; and
alert a user when the number of instruments determined based on the scannable code differs from the number of instruments determined based on the one or more images.

23. The method of claim 16, wherein the one or more processors are further used to prompt a user for confirmation that the instruments identified upon ingress or egress are counted only once.

24. The method of claim 16, further comprising displaying, on a display screen a graphical user interface with separate controls for sterile and non-sterile users.

25. The method of claim 24, further comprising providing at least two separate input devices, including at least one input device for a sterile user and at least one input device for a non-sterile user.

26. The method of claim 17,
wherein the second imaging device is configured to obtain images of the instruments with an optical filter adapted to detect hemoglobin, other body fluid, or body tissue; and
further comprising classifying, using the one or more processors, the set of instruments identified by the machine learning model during egress as used or not-used based on the presence or absence, respectively, of body fluid and tissue on the instruments.

27. The method of claim 26, further comprising generating or updating, using the one or more processors, a predictive model that predicts which instruments are likely to be used in a procedure based on a classification of instruments as used or not-used.

28. The method of claim 27, wherein the predictive model comprises an ordinal logistic regression configured to provide a probability value corresponding to the number of instruments predicted to be used in a particular surgical procedure.

29. The method of claim 27, wherein the predictive model comprises a large language model trained using a data set that includes usage data based on the classification of instruments used in a particular surgical procedure.

30. The method of claim 27, wherein the predictive model comprises a multivariate logistic regression configured to predict the number of instruments used in a particular surgical procedure.

31. A computer program product comprising one or more non-transitory computer readable media having instructions stored thereon, wherein the instructions when executed by one or more processors, cause the one or more processors to:
obtain, using at least one imaging device, one or more images of a set of the plurality of instruments upon ingress of the plurality of instruments into the sterile field;
obtain, using the at least one imaging device, one or more images of a set of the plurality of instruments upon egress of the set of the plurality of instruments from the sterile field;
generate, using the one or more processors, an ingress inventory based on the one or more images obtained upon ingress of the plurality of instruments into the sterile field;
generate, using the one or more processors, an egress inventory based on the one or more images obtained upon egress of the set of the plurality of instruments from the sterile field;
reconcile, using the one or more processors, the ingress and egress inventories by comparing the ingress and egress inventories to determine whether or not one or more instruments were left in the sterile field; and
output, using a display device, a result of the reconciliation to a user.

32. The computer program product of claim 31, wherein the at least one imaging device includes a first imaging device configured to obtain one or more images of the plurality of instruments upon ingress of the plurality of instruments into the sterile field, and a second imaging device configured to obtain one or more images of the set of the plurality of instruments upon egress of the set of the plurality of instruments from the sterile field.

33. The computer program product of claim 32, wherein the instructions, when executed by the one or more processors, further cause the one or more processors to:
identify each instrument introduced into the sterile field based on the one or more images obtained by the first imaging device upon ingress of the plurality of instruments to the sterile field; and
identify each instrument removed from the sterile field based on the one or more images obtained by the second imaging device upon egress of the set of the plurality of instruments from the sterile field.

34. The computer program product of claim 33, wherein to identify each instrument introduced and removed from the sterile field, the instructions, when executed by the one or more processors, further cause the one or more processors to:
input the one or more images obtained by the first imaging device upon ingress to a first machine learning model trained to identify instruments from image data; and
input the one or more images obtained by the second imaging device to a second machine learning model trained to identify instruments from image data.

35. The computer program product of claim 34, wherein at least one of the first and second machine learning models is trained to distinguish between visually similar instruments based on the presence or absence of a decorative discriminator applied to one or more of the instruments.

36. The computer program product of claim 32, wherein the at least one imaging device further comprises a third imaging device configured to read scannable codes carried by instruments, and instructions, when executed by the one or more processors, further cause the one or more processors to:
identify each instrument introduced into the sterile field based on a scannable code read by the third imaging device upon ingress or based on one or more images obtained by the first imaging device upon ingress; and
identify each instrument removed from the sterile field based on a scannable code read by the third imaging device upon egress or based on the one or more images obtained by the second imaging device upon egress.

37. The computer program product of claim 36, wherein the instructions, when executed by the one or more processors, further cause the one or more processors to:
determine a number of instruments in a package based on a scannable code on the package read by the third imaging device upon ingress into the sterile zone;
determine a number of instruments in the package based on one or more images obtained by the first imaging device; and
alert a user when the number of instruments determined based on the scannable code differs from the number of instruments determined based on the one or more images.

38. The computer program product of claim 31, wherein the instructions, when executed by the one or more processors, further cause the one or more processors to prompt a user for confirmation that the instruments identified upon ingress or egress are counted only once.

39. The computer program product of claim 31, wherein the instructions, when executed by the one or more processors, further cause the one or more processors todisplay, on a display screen, a graphical user interface with separate controls for sterile and non-sterile users.

40. The computer program product of claim 39, further comprising providing at least two separate input devices, including at least one input device for a sterile user and at least one input device for a non-sterile user.

41. The computer program product of claim 32,
wherein the second imaging device is configured to obtain images of the instruments with an optical filter adapted to detect hemoglobin, other body fluid, or body tissue; and
wherein the instructions, when executed by the one or more processors, further cause the one or more processors to cause the at least one processor to classify the set of instruments identified by the machine learning model during egress as used or not-used based on the presence or absence, respectively, of body fluid and tissue on the instruments.

42. The computer program product of claim 41, wherein the instructions, when executed by the one or more processors, further cause the one or more processors to generate or update a predictive model that predicts which instruments are likely to be used in a procedure based on a classification of instruments as used or not-used.

43. The computer program product of claim 42, wherein the predictive model comprises an ordinal logistic regression configured to provide a probability value corresponding to the number of instruments predicted to be used in a particular surgical procedure.

44. The computer program product of claim 42, wherein the predictive model comprises a large language model trained using a data set that includes usage data based on the classification of instruments used in a particular surgical procedure.

45. The computer program product of claim 42, wherein the predictive model comprises a multivariate logistic regression configured to predict the number of instruments used in a particular surgical procedure.
